(19) United States
(12) Patent Application Publication
(10) Pub. No.: US 2003/0017159 A1
(43) Pub. Date: Jan. 23, 2003

(54) Immunogenic tumor antigens: nucleic acids and polypeptides encoding the same and methods of use thereof

(76) Inventors: Jerome Ritz, Lincoln, MA (US); Xiao-Feng Yang, Houston, TX (US); Catherine J. Wu, Cambridge, MA (US)

Correspondence Address: Ivor R. Elrifi, Ph.D., Esq., MINTZ, LEVIN, COHN, FERRIS,, GLOVSKY and POPEO, P.C., One Financial Center, Boston, MA, 02111, US

(21) Appl. No.: 10/136,417

(22) Filed: May 1, 2002

Related U.S. Application Data
(60) Provisional application No. 60/288,068, filed on Jul. 20, 2001.
(60) Provisional application No. 60/306,982, filed on Jul. 20, 2001.
(60) Provisional application No. 60/386,178, filed on Feb. 1, 2002.

(51) Int. Cl. G01N 33/574; A61K 39/395

(52) U.S. Cl. 424/155.1; 435/7.23

(57) ABSTRACT

The invention provides human chronic myelocytic leukemia-like proteins (CML protein) and isolated nucleic acid molecules encoding the same. Also provided are antibodies that immunospecifically-bind to CML polypeptides or polynucleotides, or derivatives, variants, mutants, or fragments thereof. The invention additionally provides methods in which CML polypeptides, polynucleotides, and antibodies are used in the detection, prevention, and treatment of a broad range of pathological states, and methods of treating malignancy-related disorders by modulating activity or expression of CML proteins.

### RELATED APPLICATIONS

This application claims priority from U.S. Ser. No. 60/288,068, filed May 2, 2001; U.S. Ser. No. 60/306,982, filed Jul. 20, 2001; and U.S. Ser. No. 60/______ , filed Feb. 1, 2002, each of which is incorporated by reference in its entirety.

### FIELD OF THE INVENTION

The invention relates to generally to polypeptides and nucleic acids encoding immunogenic tumor antigens, and in particular to tumor antigens which elicit an immune response associated with the remission of chronic myelogenous leukemia.

### BACKGROUND OF THE INVENTION

The therapeutic benefit of allogeneic bone marrow transplantation (BMT) derives in part from the eradication of leukemia cells by high dose chemotherapy and radiation [1][2]. However, several clinical observations provide convincing evidence that donor immune elements also contribute substantially to the elimination of residual leukemia following BMT [3][4]. These observations include the reduced risk of relapse after BMT in patients who develop graft-versus-host disease (GVHD) and the increased risk of relapse in patients who receive T cell depleted donor marrow [4][5]. In addition, it has been demonstrated that relapse after BMT can often be successfully treated by infusion of donor lymphocytes without additional therapy [31][32]. The demonstration that adoptive immunotherapy with donor lymphocyte infusion (DLI) can provide long lasting remissions provides compelling evidence that donor T cells play an important role in mediating a graft-versus-leukemia (GVL) response as well as GVHD after allogeneic BMT [6].

Appreciation of the importance of GVL has led to the development of less intensive non-myeloablative approaches for transplantation of allogeneic hematopoietic stem cells with subsequent infusion of donor T cells to enhance anti-tumor immunity. Initial reports using these approaches are encouraging and provide evidence that the therapeutic effects of DLI can be extended to provide effective immunity against solid tumors as well as hematopoietic malignancies [7]. Furthermore, in patients with relapsed chronic myelocytic leukemia (CML) after allogeneic BMT, the infusion of donor lymphocytes initiates an effective anti-tumor response that results in the elimination of leukemia cells in over 70% of patients [33]. Although responses can be delayed in some individuals, most patients who achieve a cytogenetic response also subsequently become PCR negative for cells containing bcr-abl transcripts. These observations demonstrate that the anti-leukemia response associated with DLI results in the elimination of relatively large numbers of tumor cells and, thus far, very few patients have relapsed after achieving a molecular response. Despite the effectiveness of DLI, the target antigens of this immune response have not been well characterized.

Thus, a need remains in the art for the identification of target antigens that are immunogenic in a wide variety of malignancies and may be a good target for antigen-specific immunotherapy in different solid tumors as well as hematologic malignancies.

### SUMMARY OF THE INVENTION

The invention is based in part on the discovery of two novel tumor associated antigens, CML 28 (SEQ ID NO: 2; FIG. 2A) and CML 66 (SEQ ID NO: 4; FIG. 15). Both of these antigens, the polypeptides and polynucleotides that encode them, and any fragments or variants thereof are collectively referred to as “CML nucleic acids” or “CML polynucleotides” and the corresponding encoded polypeptides are referred to as “CML polypeptides” or “CML proteins.” Unless indicated otherwise, “CML” is meant to refer to any of the novel sequences disclosed herein.

In one aspect, the invention provides an isolated nucleic acid sequence, homologous to a gene which encodes either CML 28 or CML 66, wherein the sequence comprises SEQ ID NO: 1 and/or 3, or an allelic or substitution variant thereof. In another embodiment, there is provided an oligonucleotide that includes a portion of SEQ ID NO: 1 and/or 3.

In other aspects, the invention provides a vector comprising one or more of the isolated nucleic acid sequences or oligonucleotides described herein, and a host cell transformed with one or more vectors described herein. Also provided is a method for producing a CML 28 and/or CML 66 protein by culturing a host cell transformed with one or more vectors described herein under conditions suitable for the expression of the protein encoded by the vector.

In still another aspect, the invention provides a pharmaceutical composition that comprises an isolated nucleic acid or oligonucleotide described herein and a pharmaceutically-acceptable carrier or excipient.

In another aspect, there is provided an isolated CML 28 and/or CML 66 protein encoded by an isolated nucleic acid sequence or oligonucleotide described herein. In some aspects, the isolated protein comprises the amino acid sequence of CML28(SEQ ID NO: 2) or CML66 (SEQ ID NO: 4), or functional variants or fragments thereof. In another embodiment, a variant or fragment of the CML 28 and CML 66 proteins retain their immunogenic activity.

In yet another aspect, there is provided an antibody that binds specifically to an isolated CML 28 and/or CML 66 protein, or fragment thereof. The antibody can be a monoclonal or polyclonal antibody, or fragments and derivatives thereof, e.g., a labeled antibody.

The invention further provides a method of treating cancer in a mammal by administering at least one agent which modulates the expression or activity of CML 28 and/or CML 66. In still another embodiment, the agent which modulates either CML 28 or CML 66 protein activity or expression is an antibody which immunospecifically binds to a CML 28 or CML 66 polypeptide, an antibody which immunospecifically binds to a nucleic acid sequence encoding a CML 28 or CML 66 protein, or an antisense nucleic acid sequence complementary to a nucleic acid sequence encoding a CML 28 or CML 66 protein.

The invention further provides methods of identifying a CML 28 or CML 66 protein or nucleic acid encoding the same in a sample by contacting the sample with a compound that specifically binds to the polypeptide or nucleic acid, e.g., an antibody, and detecting complex formation, if present. Also provided are methods of identifying a compound that modulates the activity of a CML 28 or CML 66 protein by contacting the protein with a compound and determining whether the immunogenic activity of either CML 28 or CML 66 is modified.

In yet another aspect, the invention provides a method of determining the presence of or predisposition of a cancer associated with CML 28 or CML 66 in a subject, comprising the step of providing a sample from the subject and measuring the amount of a CML 28 or CML 66 protein in the subject sample. The amount of the particular protein in the subject sample is then compared to the amount of that protein in a control sample. A control sample is preferably taken from a matched individual, i.e., an individual of similar age, sex, or other general condition but who is not suspected of having a CML 28 or CML 66 protein-associated condition. Alternatively, the control sample may be taken from the subject at a time when the subject is not suspected of having a CML 28 or CML 66 protein-associated disorder.

In a further embodiment, the invention provides a method of determining the presence of or predisposition of a CML 28 or CML 66 protein-associated disorder in a subject. The method includes providing a nucleic acid sample, e.g., RNA or DNA, or both, from the subject and measuring the amount of the respective protein-encoding nucleic acid in the subject nucleic acid sample. The amount of a CML 28 or CML 66 protein-encoding nucleic acid in the subject nucleic acid is then compared to the amount of such nucleic acid in a control sample. An alteration in the amount of the particular protein-encoding nucleic acid in the sample relative to the amount of such nucleic acid in the control sample indicates the subject has a CML 28 or CML 66 protein-associated disorder.

In still another aspect, there is provided a method of treating or preventing or delaying a CML 28 or CML 66 protein-associated disorder. The method comprises administering to a subject in which such treatment or prevention or delay is desired a nucleic acid encoding a CML 28 or CML 66 protein, or an antibody specific for either, in an amount sufficient to treat, prevent, or delay the particular protein-associated disorder in the subject.

In a further aspect, the invention provides a method for modulating the activity of a CML 28 or CML 66 polypeptide by contacting a cell sample that includes the CML 28 or CML 66 polypeptide with a compound that binds to the SECX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

The polynucleotides and polypeptides are used as immunogens to produce antibodies specific for the invention, and as vaccines. They are used to screen for potential agonist and antagonist compounds. For example, a cDNA encoding CML 28 may be useful in gene therapy, and CML 28 may be useful when administered to a subject in need thereof. By way of nonlimiting example, the compositions of the present invention will have efficacy for treatment of patients suffering the diseases and disorders listed above and/or other pathologies and disorders.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIGS. 1A-1D depict the tissue expression profile of a CML 28 gene.

FIGS. 2A and 2B: FIG. 2A shows the nucleic acid (SEQ ID NO: 1) and predicted amino acid sequence (SEQ ID NO: 2) of CML 28. The putative translation initiation site is in boldface type. The 5′ end of CML28 originally cloned from a CML cDNA library is indicated by an arrow. The polyadenylation site in 3′ untranslated region is underlined. FIG. 2B shows the amino acid homology between CML28 and bacterial RNase PH (SEQ ID NO: 11). The identical amino acids are indicated with the abbreviation of the amino acid. The similar amino acids is indicated by +. A string of X's resulting from a BLAST search is a result of automatic filtering of the query for low-complexity sequence that is performed to prevent artifactual hits. The filter substitutes any low-complexity sequence that it finds the letter “X” in protein sequences (e.g., “XXX”). Low-complexity regions can result in high scores that reflect compositional bias rather than significant position-by-position alignment (Wootton and Federhen, Methods Enzymol 266:554-571, 1996). The dashed lines indicate the sequence gap in construction of the alignment.

FIG. 3 shows the human chromosome localization of CML 28.

FIG. 4 depicts the analysis of immune reactivity of CML 28 by Western blots.

FIG. 5 depicts a quantitative CML 28-specific IgG measured using ELISA in serum from normal donors, patients with either CML, lung cancer, melanoma or prostate cancer.

FIG. 6 shows the correlation of CML 28-specific IgG with a cytogenetic response following donor lymphocyte infusion.

FIG. 7 shows the correlation of CML 28-specific IgG with a cytogenetic response following donor lymphocyte infusion.

FIG. 8 shows the distribution of a CML 28 polypeptide in hematopoietic tissues, cell lines and primary leukemias using an anti-CML28 murine monoclonal antibody.

FIGS. 9A-9D depicts the tissue expression profile of CML 66 gene.

FIG. 10 shows the human chromosome localization of CML 66 by FISH.

FIG. 11 shows the analysis of immune reactivity of CML 66 by Western blot.

FIG. 12 shows a quantitative anti-CML 66 IgG measured using ELISA in serum from normal donors, patients with either CML, lung cancer, melanoma or prostate cancer.

FIG. 13 shows the correlation of anti-CML 66 IgG with cytogenetic response following donor lymphocyte infusion.

FIG. 14. shows the relative expression of CML 66 in normal peripheral blood mononuclear cells from normal donors (Normal) and primary CML (CML).

FIG. 15 shows the nucleic acid (SEQ ID NO: 3) and amino acid sequence (SEQ ID NO: 4) of CML66.

FIG. 16 shows single nucleotide differences in CML 66 cDNA amplified from tumor cells compared to normal CML 66 cDNA from human testis.

### DETAILED DESCRIPTION OF THE INVENTION

The reconstitution of T and B cell immunity in patients with chronic myelocytic leukemia (CML) who received infusions of CD4+ donor lymphocytes for treatment of relapse after allogeneic BMT (14) has been studied extensively. However, the identification of target antigens that would be good targets for antigen-specific immunotherapy had never been identified. Patients with CML present an ideal subject for such identification because the great majority of these patients demonstrate a complete cytogenetic and molecular response within a defined time frame after DLI and without additional intervention (15). Thus, these patients thus represent a unique opportunity to examine a consistently effective anti-tumor response in vivo. Furthermore, although T cells are presumed to be the critical mediators of GVL in these patients, previous studies have also shown that DLI initiates a complex immune response that includes a potent antibody response to a variety of leukemia-associated antigens (16).

Using established methods for serological identification of tumor antigens by recombinant cDNA expression cloning (SEREX) (17, 18) a panel of 13 leukemia-associated antigens that were recognized by high titer antibody 1 year after response to DLI were identified. Within this panel of B cell defined antigens, 11 represented known genes and 2 represented novel genes that had not previously been identified. Accordingly, the present invention is based in part on the discovery of two novel tumor associated antigens, CML 28 and CML 66.

CML28

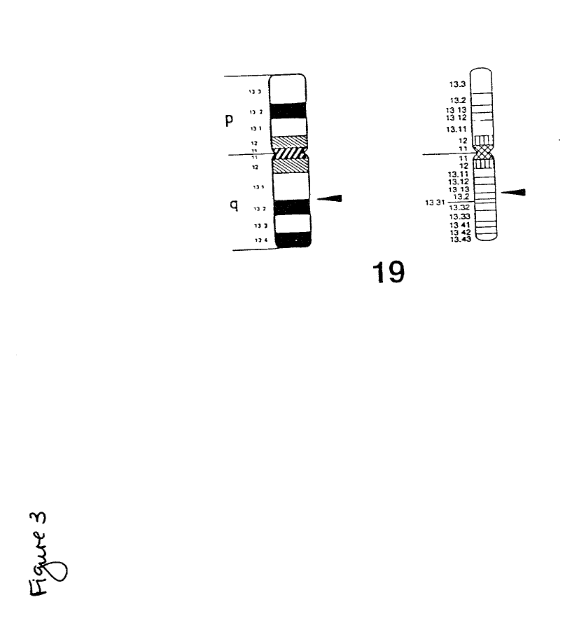

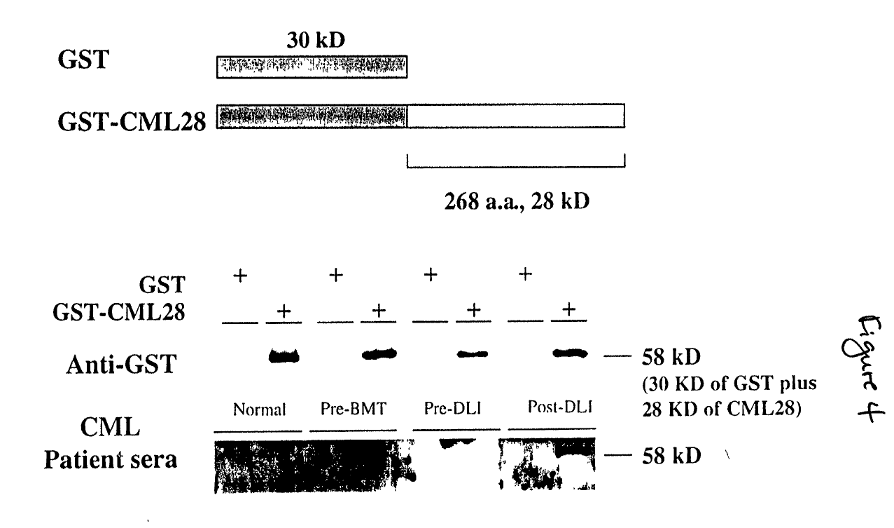

CML 28 was initially cloned from a CML expression cDNA library. A CML 28 nucleic acid (SEQ ID NO: 1) is 1126 bases in length and encodes a 268 amino acid protein (SEQ ID NO: 2) with a molecular weight of 28 kD. CML 28 shows no significant homology to other genes except a 45% homology to bacterial and yeast RNase PH, suggesting that CML 28 may be a human homolog (see, e.g., FIG. 2b and Table 1). CML 28 gene is localized to human chromosome 19q13, a region previously associated with chromosomal abnormalities in leukemia and lymphoma. CML 28 is expressed in a variety of solid tumor cell lines but high level expression in normal tissues is restricted to testis. Normal CML 28 cloned from human testis cDNA library was identical to CML 28 from the CML library. The development of high titer IgG antibody specific for CML 28 correlated with the immune induced remission of CML in two patients who received either bone marrow transplantation or infusion of normal donor lymphocytes for treatment of CML relapse. CML 28 antibody was also found in sera from 13-33% of patients with lung cancer, melanoma and prostate cancer.

CML28 gene is localized on the human chromosome 19q13. The NIH database (NCBI, NIH) indicates that in some cases of acute lymphoblastic leukemia, acute myeloid leukemia (AML) and non-Hodgkin's lymphoma, the balanced and unbalanced chromosomal abnormalities have been found in the chromosome 19q13. The Online Mendelian Inheritance in Man (OMIM) database also associates the 19q13 locus with leukemia (see, e.g., OMIM accession number 109560). These results support that chromosomal abnormalities in human chromosome 19q13 may participate in activation or upregulation of expression of CML28. Our data also showed that CML28 high expression is not only found in cultured tumor cell lines, but also found in solid tumor such prostate cancer comparing with the normal tissue from the same patient, suggesting that upregulation of CML28 may be associated with malignant transformation. Of note, for certain neoplasms, such as colorectal carcinoma, genomic hypomethylation may be responsible for the expression pattern of this category of tumor antigens [22].

Extensive search in protein databases did not yield known human proteins identical to CML28. However, CML28 shows 29% identity, 45% similarity to bacterial and yeast RNase PH based on similarity on size and overall amino acid homology. In bacteria and yeast, RNase PH plays roles in tRNA and mRNA metabolism, affection of ribosomes [34] and affection of translation of non-poly (A) mRNA [35].

Of note, CML28 expression profile is similar to that of 11 previously documented CT antigens that have been shown to express predominantly in testis and tumors. Comparing with CT antigens, CML28 has several different features: (1) CML28 has wider expression pattern in a variety of tumors [23];[22]; (2) CML28 shows a lower homology (11-19%) to other CT antigens (45-84% homology among them) [28] [27]; (4) CML28 is a single copy gene, localized in human chromosome 19q13. In contrast, some of CT antigens are multiple homologous gene families, localized in X-chromosome [29] [30].

Identification of specific antibody response in CML patients to CML28 suggested that mRNA transcripts of CML28 can be translated into a protein as an effective immunogen in eliciting immune responses. The strength of immune response to CML28 may fully depend on its expression level in the tumor, as proposed for NY-ESO-1 [20]. This argument is supported by the studies on other CT antigens such as MAGE-antigens, where data of MAGE protein expression in normal versus tumor samples by immunohistochemistry were well correlated with that by using mRNA typing [36]. Similar to the published work on MAGE antigens [31], the hybridomas secreting specific monoclonal antibody against CML28 are included in the invention.

The results shown below in Examples 1 through 6 indicate that humoral immune responses to CML28 is not associated with potential graft-versus-host diseases (GVHD) following BMT but is specifically associated with the leukemia remission process in CML patients who respond to DLI therapy, strongly suggesting that specific immune responses to CML28 may associate with antitumor immunity (GVL) in CML patients. For absence of immune responses to testis antigens including CML28 in normal individuals, the following two explanations have been proposed. Testis is believed to be an immunoprivileged site which is protected from attacks by immune reactions [32]. Alternatively, lack of HLA class I expression may also contribute to the absence of immune responses to testis antigen [33].

Normal CML28 sequence cloned from human testis cDNA library is identical to that originally cloned from CML cDNA library, suggesting that immunogenicity to CML28 may not be resulted from mutations in CML28 sequence.

Of note, most DLI responders did not have detectable reactivity to CML28 and any other 12 antigens identified in our initial DLI responders [9]. This observation suggests that the number of leukemia-associated antigens may be quite diverse. This may reflect the high degree of diversity of human HLA as well as the large number of potential targets. CML28 specific high titer IgG antibody and IgG antibody subtype switching (IgG1 and IgG4) in post-DLI serum suggested a potential T helper cell response to CML28, since previous reports showed that Th cell epitopes of certain proteins are often localized close to or within B cell epitopes and IL-4 is a major factor to induce IgG4 switching One encouraging study come from Jager et al. [20]) on NY-ESO-1, in which it is shown that CD8+ T cell response to HLA-A2-restricted NY-ESO-1 peptides were detected in 10 of 11 patients with NY-ESO-1 antibody, but not in patients lacking antibody or in patients with NY-ESO-1-negative tumor. Since NY-ESO-1 was also originally cloned by SEREX approach and is also a CT antigen, CD8+ cytotoxic T cell response specifically to CML28 is expected to be associated with CML remission in the DLI-responding patients.

In summary, the characterization of CML28 demonstrates that this novel gene is highly expressed in different solid tumors but high level expression in normal tissues is restricted to testis. Using a sensitive ELISA, the highest titers of CML specific IgG antibody were found in patients with CML who responded to either BMT or DLI. In these patients, the development of high titer specific antibody correlated well with the cytogenetic remission induced by DLI. IgG antibodies specific for CML28 were also found in 30-60% of patients with melanoma and prostate cancer. These observations indicate that CML28 antigen is immunogenic in patients with different solid tumors as well as in patients with leukemia and this response is not restricted to patients after allogeneic bone marrow transplantation. The immunogenicity of this novel antigen and association with effective anti-tumor immunity in CML suggest that CML28 may also be an appropriate target for immunotherapy in other malignancies.

CML66

CML 66 was initially cloned from a CML cDNA expression library. A disclosed CML 66 nucleic acid is 2319 bases in length (SEQ ID NO: 3) and encodes a 583 amino acid protein (SEQ ID NO: 4) with a molecular weight of 66 kD and has no significant homology to other known genes. CML 66 gene is localized to human chromosome 8q23. This locus is associated with several diseases, including glaucoma (see, e.g., OMIM accession numbers 216550; 603563; 602429; and 140300). CML 66 is expressed in acute and chronic leukemias and in a variety of solid tumor cell lines. When examined by Northern blot, expression in normal tissues is restricted to testis and heart and no expression was found in hematopoietic tissues. The development of high titer IgG antibody specific for CML 66 correlated with immune induced remission of CML in a patient who received infusion of normal donor lymphocytes for treatment of relapse. CML 66 antibody was also found in sera from 20-50% of patients with lung cancer, melanoma and prostate cancer.

These findings suggest that both CML 28 and CML 66 may be immunogenic in a wide variety of malignancies and may be a good target for antigen-specific immunotherapy in different solid tumors as well as hematologic malignancies.

CML Nucleic Acids

The novel nucleic acids provided by the invention include those that encode a CML protein, or biologically-active portions thereof. The encoded polypeptides can thus include, e.g., the amino acid sequence of CML28 (SEQ ID NO: 2) or CML66 (SEQ ID NO: 4). These sequences comprise an ORF encoding a novel CML protein of the invention, as described above.

In some embodiments, a CML nucleic acid according to the invention encodes a mature form of a CML protein. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

In some embodiments, a nucleic acid encoding a polypeptide having the amino acid sequence of a CML polypeptide includes the nucleic acid sequence of SEQ ID NO: 1 and/or 3, or a fragment, thereof. Additionally, the invention includes mutant or variant nucleic acids of SEQ ID NO: 1 and/or 3, or a fragment thereof, any of whose bases may be changed from the disclosed sequence while still encoding a protein that immunogenic—like biological activities and physiological functions. The invention further includes the complement of the nucleic acid sequence of a CML nucleic acid, e.g., SEQ ID NO: 1 and/or 3, including fragments, derivatives, analogs and homologs thereof. The invention additionally includes nucleic acids or nucleic acid fragments, or complements thereto, whose structures include chemical modifications.

Also included are nucleic acid fragments sufficient for use as hybridization probes to identify CML protein-encoding nucleic acids (e.g., CML mRNA) and fragments for use as polymerase chain reaction (PCR) primers for the amplification or mutation of CML protein nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments, and homologs thereof. The nucleic acid molecule can be single-stranded or double-stranded, but preferably is double-stranded DNA.

The term “probes” refer to nucleic acid sequences of variable length, preferably between at least about 10 nucleotides (nt), 100 nt, or as many as about, e.g., 6,000 nt, depending upon the specific use. Probes are used in the detection of identical, similar, or complementary nucleic acid sequences. Longer length probes are usually obtained from a natural or recombinant source, are highly specific and much slower to hybridize than oligomers. Probes may be single- or double-stranded, and may also be designed to have specificity in PCR, membrane-based hybridization technologies, or ELISA-like technologies.

The term “isolated” nucleic acid molecule is a nucleic acid that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated CML nucleic acid molecule can contain less than approximately 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NO: 1 and/or 3, or a complement of this nucleotide sequence, can be isolated using standard molecular biology techniques and the sequence information provided herein. Using all or a portion of the nucleic acid sequence of SEQ ID NO: 1 and/or 3 as a hybridization probe, CML protein-encoding nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to CML nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of SEQ ID NO: 1 and/or 3, or a complement thereof. Oligonucleotides may be chemically synthesized and may also be used as probes.

In another embodiment, an isolated nucleic acid molecule of the invention includes a nucleic acid molecule that is a complement of the nucleotide sequence shown in any of SEQ ID NO: 1 and/or 3. In still another embodiment, an isolated nucleic acid molecule of the invention includes a nucleic acid molecule that is a complement of the nucleotide sequence shown in any of SEQ ID NO: 1 and/or 3, or a portion of this nucleotide sequence. A nucleic acid molecule that is complementary to the nucleotide sequence shown in SEQ ID NO: 1 and/or 3 is one that is sufficiently complementary to the nucleotide sequence shown in SEQ ID NO: 1 and/or 3 that it can hydrogen bond with little or no mismatches to the nucleotide sequence shown in SEQ ID NO: 1 and/or 3, thereby forming a stable duplex.

As used herein, the term “complementary” refers to Watson-Crick or Hoogsteen base-pairing between nucleotides units of a nucleic acid molecule, whereas the term “binding” is defined as the physical or chemical interaction between two polypeptides or compounds or associated polypeptides or compounds or combinations thereof. Binding includes ionic, non-ionic, Von der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Additionally, the nucleic acid molecule of the invention can comprise only a portion of the nucleic acid sequence of any of SEQ ID NO: 1 and/or 3, e.g., a fragment that can be used as a probe or primer, or a fragment encoding a biologically active portion of a CML protein. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild-type.

Derivatives and analogs may be full-length or other than full-length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See, e.g., Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489), which is incorporated herein by reference in its entirety.

The term “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of CML polypeptide. Isoforms can be expressed in different tissues of the same organism as a result of, e.g., alternative splicing of RNA. Alternatively, isoforms can be encoded by different genes. In the invention, homologous nucleotide sequences include nucleotide sequences encoding for a CML polypeptide of species other than humans, including, but not limited to, mammals, and thus can include, e.g., mouse, rat, rabbit, dog, cat cow, horse, and other organisms. Homologous nucleotide sequences also include, but are not limited to, naturally-occurring allelic variations and mutations of the nucleotide sequences set forth herein. A homologous nucleotide sequence does not, however, include the nucleotide sequence encoding CML protein. Homologous nucleic acid sequences include those nucleic acid sequences that encode conservative amino acid substitutions (see below) in SEQ ID NO: 1 and/or 3, as well as a polypeptide having immunogenic-like activity, as described above. A homologous amino acid sequence does not encode the amino acid sequence of a CML protein.

The nucleotide sequence disclosed for the CML protein gene allows for the generation of probes and primers designed for use in identifying CML protein-expressing cell types, e.g. liver cells, and/or cloning CML protein homologues in other cell types, e.g., from other tissues, as well as CML protein homologues from other mammals. The probe/primer typically includes a substantially-purified oligonucleotide. The oligonucleotide typically includes a region of nucleotide sequence that hybridizes under stringent conditions to at least about 12, 25, 50, 100, 150, 200, 250, 300, 350 or 400 or more consecutive sense strand nucleotide sequence of a CML nucleic acid, e.g., one including all or a portion of SEQ ID NO: 1 and/or 3. Alternatively, the oligonucleotide sequence may include a region of nucleotide sequences that hybridizes to some or all of an anti-sense strand of a strand encoding CML nucleic acid. For example, the oligonucleotide may include some or all of the anti-sense strand nucleotide sequence of SEQ ID NO: 1 and/or 3, or of a naturally occurring mutant of one of these nucleic acids.

Probes based upon the CML nucleotide sequence can be used to detect transcripts or genomic sequences encoding the same or homologous proteins. In various embodiments, the probe further includes a label group attached thereto, e.g., the label group can be a radioisotope, a fluorescent compound, an enzyme, or an enzyme co-factor. Such probes can be used as a part of a diagnostic test kit for identifying cells or tissue (e.g., liver) which mis-express a CML protein, such as by measuring a level of a CML protein-encoding nucleic acid in a sample of cells from a subject e.g., detecting CML mRNA levels or determining whether a genomic CML gene has been mutated or deleted.

The term “a polypeptide having a biologically-active portion of CML protein” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of CML protein” can be prepared by isolating a portion of a nucleotide, e.g., a nucleotide including a portion of SEQ ID NO: 1 and/or 3, that encodes a polypeptide having immunogenic-like biological activity (as described above), expressing the encoded portion of CML protein (e.g., by recombinant expression in vitro) and assessing the activity of the encoded portion of CML protein.

CML Nucleic Acid Variants

The invention further encompasses nucleic acid molecules that differ from the disclosed CML nucleotide sequence due to degeneracy of the genetic code. These nucleic acids can encode the same CML protein as those encoded by the nucleotide sequence of SEQ ID NO: 1 and/or 3. In another embodiment, an isolated nucleic acid molecule of the invention has a nucleotide sequence encoding a protein having the amino acid sequence of CML28 (SEQ ID NO: 2) or CML66 (SEQ ID NO: 4).

In addition to the CML nucleotide sequence shown in SEQ ID NO: 1 and/or 3 it will be appreciated by those skilled in the art that DNA sequence polymorphisms that lead to changes in the amino acid sequence of CML protein may exist within a population (e.g., the human population). Such genetic polymorphism in the CML protein gene may exist among individuals within a population due to natural allelic variation. As used herein, the terms “gene” and “recombinant gene” refer to nucleic acid molecules comprising an open reading frame encoding a CML protein, preferably a mammalian protein. Such natural allelic variations can typically result in 1-5% variance in the nucleotide sequence of the CML gene. Any and all such nucleotide variations and resulting amino acid polymorphisms in CML protein that are the result of natural allelic variation and that do not alter the functional activity of CML protein are intended to be within the scope of the invention.

Additionally, nucleic acid molecules encoding CML protein proteins from other species, and thus that have a nucleotide sequence that differs from the nucleic acid sequence of CML protein (e.g., it differs from SEQ ID NO: 1 and/or 3), are intended to be within the scope of the invention. Nucleic acid molecules corresponding to natural allelic variants and homologues of the CML cDNAs of the invention can be isolated based on their homology to the CML protein-encoding nucleic acids disclosed herein using the human cDNAs, or a portion thereof, as a hybridization probe according to standard hybridization techniques under stringent hybridization conditions.

In another embodiment, an isolated nucleic acid molecule of the invention is at least 6 nucleotides in length and hybridizes under stringent conditions to the nucleic acid molecule comprising the nucleotide sequence of a CML nucleic acid, e.g., SEQ ID NO: 1 and/or 3. In another embodiment, the nucleic acid is at least 10, 25, 50, 100, 250, 500 or 750 nucleotides in length. In yet another embodiment, an isolated nucleic acid molecule of the invention hybridizes to the coding region. As used herein, the term “hybrdizes under stringent conditions” is intended to describe conditions for hybridization and washing under which nucleotide sequences at least 60% homologous to each other typically remain hybridized to each other.

Homologs (i.e., nucleic acids encoding CML protein derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present at excess, at Tₘ, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of a CML nucleic acid, including those described herein, corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of a CML nucleic acid (e.g. SEQ ID NO: 1 and/or 3), or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al., (eds.), 1993, CURRENT PROTOCOLS IN MOLECULAR BIOLOGY, John Wiley & Sons, NY, and Kriegler, 1990. GENE TRANSFER AND EXPRESSION, A LABORATORY MANUAL, Stockton Press, NY.

In a third embodiment, a nucleic acid that is hybridizable to the nucleic acid molecule comprising the nucleotide sequence of a CML nucleic acid (e.g., it hybridizes to SEQ ID NO: 1 and/or 3), or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al., (eds.), 1993. CURRENT PROTOCOLS IN MOLECULAR BIOLOGY, John Wiley & Sons, NY, and Kriegler, 1990. GENE TRANSFER AND EXPRESSION, A LABORATORY MANUAL, Stockton Press, NY; Shilo and Weinberg, 1981. Proc. Natl. Acad. Sci. USA 78: 6789-6792.

Conservative Mutations

In addition to naturally-occuring allelic variants of the CML protein-encoding sequence that may exist in the population, the skilled artisan will further appreciate that changes can be introduced by mutation into the nucleotide sequence of a CML nucleic acid (e.g., SEQ ID NO: 1 and/or 3), thereby leading to changes in the amino acid sequence of the encoded CML protein, without altering the functional ability of the protein. For example, nucleotide substitutions leading to amino acid substitutions at “non-essential” amino acid residues can be made in the sequence of SEQ ID NO: 1 and/or 3. A “non-essential” amino acid residue is a residue that can be altered from the wild-type sequence of CML protein without altering the biological activity, whereas an “essential” amino acid residue is required for biological activity.

Another aspect of the invention pertains to nucleic acid molecules encoding CML protein that contain changes in amino acid residues that are not essential for activity. Such proteins differ in amino acid sequence from the amino acid sequence of a CML protein (e.g., CML28 (SEQ ID NO: 2) or CML66 (SEQ ID NO: 4)), yet retain biological activity. In one embodiment, the isolated nucleic acid molecule includes a nucleotide sequence encoding a protein, wherein the protein includes an amino acid sequence at least about 75% homologous to the amino acid sequence of any of CML28 (SEQ ID NO: 2) or CML66 (SEQ ID NO: 4). Preferably, the protein encoded by the nucleic acid is at least about 80% homologous to any of CML28 (SEQ ID NO: 2) or CML66 (SEQ ID NO: 4), more preferably at least about 90%, 95%, 98%, and most preferably at least about 99% homologous to CML28 (SEQ ID NO: 2) or CML66 (SEQ ID NO: 4).

An isolated nucleic acid molecule encoding a CML protein homologous to a CML protein, e.g., a polypeptide including the amino acid sequence of any of CML28 (SEQ ID NO: 2) or CML66 (SEQ ID NO: 4), can be created by introducing one or more nucleotide substitutions, additions or deletions into the corresponding CML nucleotide sequence, such that one or more amino acid substitutions, additions or deletions are introduced into the encoded protein.

Mutations can be introduced into CML protein-encoding nucleic acid by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Thus, a predicted nonessential amino acid residue in CML protein is replaced with another amino acid residue from the same side chain family. Alternatively, in another embodiment, mutations can be introduced randomly along all or part of a CML protein coding sequence, such as by saturation mutagenesis, and the resultant mutants can be screened for CML protein biological activity to identify mutants that retain activity. Following mutagenesis of the CML nucleic acid, the encoded protein can be expressed by any recombinant technology known in the art and the activity of the protein can be determined.

In one embodiment, a mutant CML protein can be assayed for: (i) the ability to form protein:protein interactions with other CML proteins, other cell-surface proteins, or biologically-active portions thereof; (ii) complex formation between a mutant CML protein and a CML protein receptor; (iii) the ability of a mutant CML protein to bind to an intracellular target protein or biologically active portion thereof, or (iv) the ability to specifically bind an anti-CML protein antibody.

Antisense Nucleic Acids

Another aspect of the invention pertains to isolated antisense nucleic acid molecules that are hybridizable to or complementary to the nucleic acid molecule including a CML nucleic acid (e.g., a nucleic acid including SEQ ID NO: 1 and/or 3), or fragments, analogs or derivatives thereof. An “antisense” nucleic acid includes a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire CML protein coding strand, or to only a portion thereof.

In one embodiment, an antisense nucleic acid molecule is antisense to a “coding region” of the coding strand of a nucleotide sequence encoding CML protein. The term “coding region” refers to the region of the nucleotide sequence comprising codons which are translated into amino acid residues (e.g., SEQ ID NO: 1 and/or 3). In another embodiment, the antisense nucleic acid molecule is antisense to a “non-coding region” of the coding strand of a CML nucleotide sequence. The term “non-coding region” refers to 5′ and 3′ sequences which flank the coding region that are not translated into amino acids (i.e., also referred to as 5′ and 3′ non-translated regions).

Given the coding strand sequences encoding CML protein disclosed herein, antisense nucleic acids of the invention can be designed according to the rules of Watson and Crick or Hoogsteen base-pairing. The antisense nucleic acid molecule can be complementary to the entire coding region of CML protein mRNA, but more preferably is an oligonucleotide that is antisense to only a portion of the coding or non-coding region of CML protein mRNA. For example, the antisense oligonucleotide can be complementary to the region surrounding the translation start site of CML protein mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids, e.g., phosphorothioate derivatives and acridine-substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a CML protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual α-units, the strands run parallel to each other (Gaultier, et al., 1987. Nucl. Acids Res. 15: 6625-6641). The antisense nucleic acid molecule can also comprise a 2′-o-methylribonucleotide (Inoue, et al., 1987. Nucl. Acids Res. 15: 6131-6148) or a chimeric RNA-DNA analogue (Inoue, et al., 1987. FEBS Lett. 215: 327-330).

Ribozymes and PNA Moieties

Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes; described by Haselhoff and Gerlach, 1988. Nature 334: 585-591) can be used to catalytically-cleave CML protein mRNA transcripts to thereby inhibit translation of CML protein mRNA. A ribozyme having specificity for a CML nucleic acid can be designed based upon the nucleotide sequence of CML protein DNA disclosed herein (e.g., SEQ ID NO: 1 and/or 3). For example, a derivative of a Tetrahymena L-19 IVS RNA can be constructed in which the nucleotide sequence of the active site is complementary to the nucleotide sequence to be cleaved in a CML protein-encoding mRNA. See, e.g., Cech, et al., U.S. Pat. No. 4,987,071; and Cech, et al., U.S. Pat. No. 5,116,742 Alternatively, CML protein mRNA can be used to select a catalytic RNA having a specific ribonuclease activity from a pool of RNA molecules (Bartel, et al., 1993. Science 261: 1411-1418).

Alternatively, CML protein gene expression can be inhibited by targeting nucleotide sequences complementary to the regulatory region of the CML nucleic acid (e.g., the promoter and/or enhancers) to form triple helical structures that prevent transcription of the CML protein gene in target cells. See, e.g., Helene, 1991. Anticancer Drug Des. 6: 569-84; Helene, et al., 1992. Ann. N.Y. Acad. Sci. 660: 27-36; and Maher, 1992. Bioassays 14: 807-15.

In various embodiments, the nucleic acids of CML protein can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (Hyrup, et al., 1996. Bioorg. Med. Chem. 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. above; Perry-O'Keefe, et al., 1996. Proc. Natl. Acad. Sci. USA 93: 14670-14675.

PNAs of CML can be used in therapeutic and diagnostic applications. For example, PNAs can be used as antisense or antigene agents for sequence-specific modulation of gene expression by, e.g., inducing transcription or translation arrest or inhibiting replication. PNAs of CML can also be used, e.g., in the analysis of single base pair mutations in a gene by, e.g., PNA directed PCR clamping; as artificial restriction enzymes when used in combination with other enzymes, e.g., S1 nucleases (see, Hyrup, 1996., above); or as probes or primers for DNA sequence and hybridization (see, Hyrup, et al., 1996.; Perry-O'Keefe, 1996., above).

In another embodiment, PNAs of CML can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of CML can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, 1996., above). The synthesis of PNA-DNA chimeras can be performed as described in Finn, et al., (1996. Nucl. Acids Res. 24: 3357-3363). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag, et al., 1989. Nucl. Acid Res. 17: 5973-5988). PNA monomers are then coupled in a stepwise manner to produce a chimeric molecule with a 5′ PNA segment and a 3′ DNA segment (see, Finn, et al., 1996., above). Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO 88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucdeotide may be conjugated to another molecule, e.g., a peptide, a hybridization triggered cross-linking agent, a transport agent, a hybridization-triggered cleavage agent, and the like.

CML Polypeptides

A polypeptide according to the invention includes a polypeptide including the amino acid sequence of a CML polypeptide (e.g., CML28 or CML66). In some embodiments, the CML polypeptide includes the amino acid sequence of either SEQ ID NO: 2 or 4. In various embodiments, a CML polypeptide is provided in a form longer than the sequence of the mature CML protein. For example, the polypeptide may be provided as including an amino terminal signal sequence. In other embodiments, the CML polypeptide is provided as the mature form of the polypeptide.

The invention also includes a mutant or variant protein any of whose residues may be changed from the corresponding residues shown in either CML28 (SEQ ID NO: 2) or CML66 (SEQ ID NO: 4), while still encoding a protein that maintains its immunogenic-like activities and physiological functions, or a functional fragment thereof.

In general, a CML protein variant that preserves immunogenic-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to an isolated CML protein, as described above, and biologically-active portions thereof, or derivatives, fragments, analogs or homologs thereof. Also provided are polypeptide fragments suitable for use as immunogens to raise anti-CML protein antibodies. In one embodiment, native CML protein can be isolated from cells or tissue sources by an appropriate purification scheme using standard protein purification techniques. In another embodiment, CML protein is produced by recombinant DNA techniques. Alternative to recombinant expression, CML protein or polypeptide can be synthesized chemically using standard peptide synthesis techniques.

An “purified” polypeptide or protein or biologically-active portion thereof is substantially free of cellular material or other contaminating proteins from the cell or tissue source from which the CML protein is derived, or substantially free from chemical precursors or other chemicals when chemically synthesized. The language “substantially free of cellular material” includes preparations of CML protein in which the protein is separated from cellular components of the cells from which it is isolated or recombinantly-produced. In one embodiment, the language “substantially free of cellular material” includes preparations of CML protein having less than about 30% (by dry weight) of a non-CML protein (also referred to herein as a “contaminating protein”), more preferably less than about 20% of a contaminating protein, still more preferably less than about 10% of a contaminating protein, and most preferably less than about 5% of a contaminating protein. When the CML protein or biologically-active portion thereof is recombinantly-produced, it is also preferably substantially free of culture medium, i.e., culture medium represents less than about 20%, more preferably less than about 10%, and most preferably less than about 5% of the volume of the CML protein preparation.

The phrase “substantially free of chemical precursors or other chemicals” includes preparations of CML protein in which the protein is separated from chemical precursors or other chemicals that are involved in the synthesis of the protein. In one embodiment, the language “substantially free of chemical precursors or other chemicals” includes preparations of CML protein having less than about 30% (by dry weight) of chemical precursors or non-CML chemicals (also referred to herein as “chemical contaminants”), more preferably less than about 20% chemical contaminants, still more preferably less than about 10% chemical contaminants, and most preferably less than about 5% chemical contaminants.

Biologically-active portions of a protein include peptides comprising amino acid sequences sufficiently homologous to or derived from the amino acid sequence of the CML protein which include fewer amino acids than the full-length protein, and exhibit at least one activity of a CML protein. Typically, biologically-active portions comprise a domain or motif with at least one activity of the CML protein. A biologically-active portion of a CML protein can be a polypeptide which is, for example, 10, 25, 50, 100 or more amino acids in length.

A biologically-active portion of the CML protein of the invention may contain at least one of the above-identified conserved domains. Moreover, other biologically active portions, in which other regions of the protein are deleted, can be prepared by recombinant techniques and evaluated for one or more of the functional activities of a native CML protein.

In some embodiments, the CML protein has a sequence which is substantially homologous to CML28 (SEQ ID NO: 2) or CML66 (SEQ ID NO: 4), and retains the functional activity of the protein, yet differs in amino acid sequence due to natural allelic variation or mutagenesis, as described in detail below. Accordingly, in another embodiment, the CML protein is a protein that includes an amino acid sequence at least about 45% homologous, and more preferably about 55, 65, 70, 75, 80, 85, 90, 95, 98 or even 99% homologous to the amino acid sequence of SEQ ID NO: 2 or 4, and retains the functional activity of the corresponding CML protein having the sequence of SEQ ID NO: 2 or 4.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J. Mol. Biol. 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOs: 1, 3, 5, 7, 9, 11, 13, 15, 17, 19, 21, or 23.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide includes a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides CML protein chimeric or fusion proteins. As used herein, a CML “chimeric protein” or “fusion protein” includes a CML polypeptide operatively-linked to a non-CML polypeptide. An “CML protein or polypeptide” refers to a polypeptide having an amino acid sequence corresponding to a CML protein shown in, e.g., CML28 (SEQ ID NO: 2) or CML66 (SEQ ID NO: 4). A “non-CML polypeptide” or “non-CML protein” refers to a polypeptide having an amino acid sequence corresponding to a protein that is not substantially homologous to a CML polypeptide (e.g., a protein that is different from the CML protein and that is derived from the same or a different organism). Within a CML fusion protein the CML polypeptide can correspond to all or a portion of CML protein. In one embodiment, the fusion protein includes at least one biologically-active portion of a CML protein. In another embodiment, the fusion protein comprises at least two biologically-active portions of a CML protein. In yet another embodiment, a CML fusion protein comprises at least three biologically-active portions of a CML protein. Within the fusion protein, the term “operatively-linked” is intended to indicate that the CML polypeptide and the non-CML polypeptide are fused in-frame with one another. The non-CML polypeptide can be fused to the amino-terminus or carboxyl-terminus of the CML polypeptide.

In one embodiment, the fusion protein is a GST-CML fusion protein in which the CML sequence is fused to the carboxyl-terminus of the GST (glutathione S-transferase) sequence. Such fusion proteins can facilitate the purification of recombinant CML proteins or polypeptides.

In another embodiment, the fusion protein is a CML protein containing a heterologous signal sequence at its amino-terminus. In certain host cells (e.g., mammalian host cells), expression and/or secretion of CML protein can be increased through use of a heterologous signal sequence.

In yet another embodiment, the fusion protein is a CML-immunoglobulin fusion protein in which the CML sequence is fused to a sequence derived from a member of the immunoglobulin protein family. The CML-immunoglobulin fusion proteins of the invention can be incorporated into pharmaceutical compositions and administered to a subject to inhibit an interaction between a CML ligand and a CML protein on the surface of a cell, to thereby suppress CML protein-mediated signal transduction in vivo. The immunoglobulin fusion proteins can be used to affect the bioavailability of a CML protein cognate ligand. Inhibition of the ligand/interaction may be useful therapeutically for both the treatment of proliferative and differentiative disorders, as well as modulating (e.g., promoting or inhibiting) cell survival. Moreover, the CML-immunoglobulin fusion proteins of the invention can be used as immunogens to produce anti-CML protein antibodies in a subject, to purify CML ligands, and in screening assays to identify molecules that inhibit the interaction of CML protein with a ligand.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and re-amplified to generate a chimeric gene sequence (see, e.g., Ausubel, et al., (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A CML protein-encoding nucleic acid can be cloned into such an expression vector such that the fusion moiety is linked in-frame to the CML protein.

CML Protein Agonists and Antagonists

The invention also pertains to variants of a CML protein that function as either CML protein agonists (i.e., mimetics) or as CML protein antagonists. Variants of the CML protein can be generated by mutagenesis (e.g., discrete point mutation or truncation of the protein). An agonist of a CML protein can retain substantially the same, or a subset of, the biological activities of the naturally-occurring form of a CML protein. An antagonist of a CML protein can inhibit one or more of the activities of the naturally occurring form of the protein by, for example, competitively binding to a downstream or upstream member of a cellular signaling cascade which includes the CML protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the CML protein.

Variants of the CML protein that function as either agonists (i.e., mimetics) or as antagonists can be identified by screening combinatorial libraries of mutants (e.g., truncation mutants) of the CML protein for CML protein agonist or antagonist activity. In one embodiment, a variegated library of variants is generated by combinatorial mutagenesis at the nucleic acid level and is encoded by a variegated gene library. A variegated library of CML protein variants can be produced by, for example, enzymatically-ligating a mixture of synthetic oligonucleotides into gene sequences such that a degenerate set of potential CML protein sequences is expressible as individual polypeptides, or alternatively, as a set of larger fusion proteins (e.g., for phage display) containing the set of CML protein sequences therein. There are a variety of methods which can be used to produce libraries of potential variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CML protein sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the CML protein coding sequence can be used to generate a variegated population of fragments for screening and subsequent selection of variants of a CML protein. In one embodiment, a library of coding sequence fragments can be generated by treating a double-stranded PCR fragment of a CML coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes amino-terminal and internal fragments of various sizes of the CML protein.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CML protein. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify CML protein variants. See, e.g., Arkin and Yourvan, 1992. Proc. Natl. Acad. Sci. USA 89: 7811-7815; Delgrave, et al., 1993. Protein Engineering 6:327-331.

Anti-CML Protein Antibodies

The invention encompasses antibodies and antibody fragments, such as F_{ab} or (F_{ab})₂, that bind immunospecifically to a CML protein or polypeptide of the invention.

An isolated CML protein, or a portion or fragment thereof, can be used as an immunogen to generate antibodies that bind to CML polypeptides using standard techniques for polyclonal and monoclonal antibody preparation. The full-length CML protein can be used or, alternatively, the invention provides antigenic peptide fragments of the proteins for use as immunogens. The antigenic peptides comprise at least 4 amino acid residues of a CML polypeptide, e.g., the amino acid sequence of CML28 (SEQ ID NO: 2) or CML66 (SEQ ID NO: 4), and encompasses an epitope of CML protein such that an antibody raised against the peptide forms a specific immune complex with the protein. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of CML protein that is located on the surface of the protein (e.g., a hydrophilic region). As a means for targeting antibody production, hydropathy plots showing regions of hydrophilicity and hydrophobicity may be generated by any method well known in the art, including, for example, the Kyte-Doolittle or the Hopp-Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

CML protein sequences including, e.g., CML28 (SEQ ID NO: 2) or CML66 (SEQ ID NO: 4), or derivatives, fragments, analogs, or homologs thereof, may be used as immunogens in the generation of antibodies that immunospecifically-bind these protein components. The term “antibody” as used herein refers to immunoglobulin molecules and immunologically-active portions of inununoglobulin molecules, i.e., molecules that contain an antigen binding site that specifically-binds (i.e., immunoreacts with) an antigen, such as CML protein. Such antibodies include, but are not limited to, polyclonal, monoclonal, chimeric, single chain, F_{ab} and F_{(ab′)2} fragments, and an F_{ab} expression library. In a specific embodiment, antibodies to CML protein are disclosed. Various procedures known within the art may be used for the production of polyclonal or monoclonal antibodies to a CML protein sequence, e.g. CML28 (SEQ ID NO: 2) or CML66 (SEQ ID NO: 4), or a derivative, fragment, analog, or homolog thereof.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed CML protein or a chemically-synthesized CML polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against CML protein can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of a CML protein. A monoclonal antibody composition thus typically displays a single binding affinity for a particular CML protein with which it immunoreacts. For preparation of monoclonal antibodies directed towards a particular CML protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp.77-96). Each of the above citations is incorporated herein by reference in their entirety.

According to the invention, techniques can be adapted for the production of single-chain antibodies specific to a CML protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for a CML protein or derivatives, fragments, analogs or homologs thereof. Non-human antibodies can be “humanized” by techniques well-known within the art. See, e.g., U.S. Pat. No. 5,225,539. Antibody fragments that contain the idiotypes to a CML protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-CML protein antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al, 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314:446-449; Shaw, et al., 1988. J. Natl. Cancer Inst. 80: 1553-1559); Morrison(1985) Science 229:1202-1207; Oi, et al., (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a CML protein is facilitated by generation of hybridomas that bind to the fragment of the protein possessing such a domain. Thus, antibodies that are specific for a desired domain within a CML protein, or derivatives, fragments, analogs or homologs thereof, are also provided herein.

Anti-CML protein antibodies may be used in methods known within the art relating to the localization and/or quantitation of the protein (e.g., for use in measuring levels of the CML protein within appropriate physiological samples, for use in diagnostic methods, for use in imaging the protein, and the like). In a given embodiment, antibodies for a protein of the invention, or derivatives, fragments, analogs or homologs thereof, that contain the antibody derived binding domain, are utilized as pharmacologically-active compounds (hereinafter “Therapeutics”).

An anti-CML protein antibody (e.g., monoclonal antibody) can be used to isolate an CML polypeptide by standard techniques, such as affinity chromatography or immunoprecipitation. An anti-CML protein antibody can facilitate the purification of natural CML polypeptide from cells and of recombinantly-produced polypeptide expressed in host cells. Moreover, an anti-CML protein antibody can be used to detect CML protein (e.g. in a cellular lysate or cell supernatant) in order to evaluate the abundance and pattern of expression of the protein. Anti-CML protein antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Recombinant Expression Vectors and Host Cells

Another aspect of the invention pertains to vectors, preferably expression vectors, containing a nucleic acid encoding CML protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably, as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The phrase “regulatory sequence” is intended to includes promoters, enhancers and other expression control elements (e.g., polyadenylation signals) Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., CML proteins, mutants, fusion proteins, etc.).

The recombinant expression vectors of the invention can be designed for expression of CML protein in prokaryotic or eukaryotic cells. For example, proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T₇ promoter regulatory sequences and T₇ polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xₐ, thrombin, and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion Escherichia coli expression vectors include pTrc (Amrann et al., (1988) Gene 69:301-315) and pET 11d (Studier, et al., GENE EXPRESSION TECHNOLOGY: METHODS IN ENZYMOLOGY 185, Academic Press, San Diego, Calif. (1990) 60-89).

One strategy to maximize recombinant protein expression in Escherichia coli is to express the protein in a host bacteria with an impaired capacity to proteolytically-cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in Escherichia coli (see, e.g., Wada, et al, 1992. Nucl. Acids Res. 20: 2111-2118). Such alteration of nucleic acid sequences of the invention can be carried out by standard DNA synthesis techniques.

In another embodiment, the CML protein expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, CML protein can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid), e.g., liver cells. Tissue-specific regulatory elements are known in the art. Non-limiting examples of suitable tissue-specific promoters include the albumin promoter (liver-specific; see, Pinkert, et al., 1987. Genes Dev. 1: 268-277), lymphoid-specific promoters (see, Calame and Eaton, 1988. Adv. Immunol. 43: 235-275), in particular promoters of T cell receptors (see, Winoto and Baltimore, 1989. EMBO J. 8: 729-733) and immunoglobulins (see, Baneiji, et al., 1983. Cell 33: 729-740; Queen and Baltimore, 1983. Cell 33: 741-748), neuron-specific promoters (e.g., the neurofilament promoter; see, Byrne and Ruddle, 1989. Proc. Natl. Acad. Sci. USA 86: 5473-5477), pancreas-specific promoters (see, Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (see, Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to CML mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, CML protein can be expressed in bacterial cells such as Escherichia coli, insect cells, yeast or mammalian cells (such as Chinese hamster ovary cells ((CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al., (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding CML protein or can be introduced on a separate vector. Cells stably-transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) CML protein. Accordingly, the invention further provides methods for producing CML protein using the host cells of the invention. In one embodiment, the method comprises culturing the host cell of invention (i.e., into which a recombinant expression vector encoding CML protein has been introduced) in a suitable medium such that CML protein is produced. In another embodiment, the method further comprises isolating the protein from the medium or the host cell.

Transgenic Animals

The host cells of the invention can also be used to produce non-human transgenic animals. For example, in one embodiment, a host cell of the invention is a fertilized oocyte or an embryonic stem cell into which CML protein-coding sequences have been introduced. These host cells can then be used to create non-human transgenic animals in which exogenous CML nucleic acids sequences have been introduced into their genome or homologous recombinant animals in which endogenous CML sequences have been altered. Such animals are useful for studying the function and/or activity of CML protein and for identifying and/or evaluating modulators of the protein's activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc.

A transgene is exogenous DNA that is integrated into the genome of a cell from which a transgenic animal develops and that remains in the genome of the mature animal, thereby directing the expression of an encoded gene product in one or more cell types, e.g., liver, or tissues of the transgenic animal. As used herein, a “homologous recombinant animal” is a non-human animal, preferably a mammal, more preferably a mouse, in which an endogenous CML protein gene has been altered by homologous recombination between the endogenous gene and an exogenous DNA molecule introduced into a cell of the animal, e.g., an embryonic cell of the animal, prior to development of the animal.

A transgenic animal of the invention can be created by introducing CML protein-encoding nucleic acid into the male pronuclei of a fertilized oocyte (e.g., by micro-injection, retroviral infection) and allowing the oocyte to develop in a pseudopregnant female foster animal. The CML protein DNA sequence, e.g., SEQ ID NO: 1 and/or 3, can be introduced as a transgene into the genome of a non-human animal. Alternatively, a non-human homologue of the CML protein gene, such as a mouse CML protein gene, can be isolated based on hybridization to the human gene DNA and used as a transgene. Intronic sequences and polyadenylation signals can also be included in the transgene to increase the efficiency of expression of the transgene. A tissue-specific regulatory sequence(s) can be operably-linked to the CML protein transgene to direct expression of the protein to particular cells, e.g., liver cells. Methods for generating transgenic animals via embryo manipulation and micro-injection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the CML protein transgene in its genome and/or expression of CML mRNA in tissues or cells of the animals. A transgenic founder animal can then be used to breed additional animals carrying the transgene. Moreover, transgenic animals carrying a transgene-encoding CML protein can further be bred to other transgenic animals carrying other transgenes.

To create a homologous recombinant animal, a vector is prepared which contains at least a portion of a CML protein gene into which a deletion, addition or substitution has been introduced to thereby alter, e.g., functionally disrupt, the CML gene. The CML protein gene can be a human gene (e.g., SEQ ID NO: 1 and/or 3), but more preferably is a non-human homologue of a CML protein gene. For example, a mouse homologue of CML protein gene can be used to construct a homologous recombination vector suitable for altering an endogenous CML protein gene in the mouse genome. In one embodiment, the vector is designed such that, upon homologous recombination, the endogenous CML protein gene is functionally disrupted (i.e., no longer encodes a functional protein; also referred to as a “knock out” vector).

Alternatively, the vector can be designed such that, upon homologous recombination, the endogenous CML protein gene is mutated or otherwise altered but still encodes functional protein (e.g., the upstream regulatory region can be altered to thereby alter the expression of the endogenous CML protein). In the homologous recombination vector, the altered portion of the CML gene is flanked at its 5′- and 3′-termini by additional nucleic acid of the CML gene to allow for homologous recombination to occur between the exogenous CML gene carried by the vector and an endogenous CML gene in an embryonic stem cell. The additional flanking CML protein nucleic acid is of sufficient length for successful homologous recombination with the endogenous gene. Typically, several kilobases (Kb) of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced CML gene has homologously-recombined with the endogenous CML gene are selected. See, e.g., Li, et al., 1992. Cell 69: 915.

The selected cells are then micro-injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-human animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The nucleic acid molecules, CML proteins, and anti-CML protein antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically-acceptable carrier. As used herein, “pharmaceutically-acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and other non-aqueous (i.e., lipophilic) vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a CML protein or anti-CML protein antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The nucleic acid molecules, proteins, protein homologues, and antibodies described herein can be used in one or more of the following methods: (A) screening assays; (B) detection assays (e.g., chromosomal mapping, cell and tissue typing, forensic biology), (C) predictive medicine (e.g., diagnostic assays, prognostic assays, monitoring clinical trials, and pharmacogenomics); and (D) methods of treatment (e.g., therapeutic and prophylactic).

The isolated nucleic acid molecules of the present invention can be used to express CML protein (e.g., via a recombinant expression vector in a host cell in gene therapy applications), to detect CML mRNA (e.g., in a biological sample) or a genetic lesion in a CML protein gene, and to modulate CML protein activity, as described further, below. In addition, the CML proteins can be used to screen drugs or compounds that modulate the protein activity or expression as well as to treat disorders characterized by insufficient or excessive production of a CML protein or production of CML protein forms that have decreased or aberrant activity compared to wild-type protein. In addition, the anti-CML protein antibodies of the present invention can be used to detect and isolate proteins and modulate CML protein activity.

The invention further pertains to novel agents identified by the screening assays described herein and uses thereof for treatments as described, above.

Screening Assays

The invention provides a method (also referred to herein as a “screening assay”) for identifying modulators, i.e., candidate or test compounds or agents (e.g., peptides, peptidomimetics, small molecules or other drugs) that bind to CML protein or have a stimulatory or inhibitory effect on, e.g., CML protein expression or CML protein activity, e.g., in liver cells. The invention also includes compounds identified in the screening assays described herein.

In one embodiment, the invention provides assays for screening candidate or test compounds which bind to or modulate the activity of the membrane-bound form of a CML protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead, one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al, 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of CML protein, or a biologically-active portion thereof, on the cell surface is contacted with a test compound and the ability of the test compound to bind to a CML protein determined. The cell, for example, can of mammalian origin or a yeast cell. Determining the ability of the test compound to bind to the CML protein can be accomplished, for example, by coupling the test compound with a radioisotope or enzymatic label such that binding of the test compound to the CML protein or biologically-active portion thereof can be determined by detecting the labeled compound in a complex. For example, test compounds can be labeled with ¹²⁵I, ³⁵s, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of CML protein, or a biologically-active portion thereof, on the cell surface with a known compound which binds CML protein to form an assay mixture, contacting the assay mixture with a test compound, and determining the ability of the test compound to interact with a CML protein, wherein determining the ability of the test compound to interact with the protein comprises determining the ability of the test compound to preferentially bind to CML protein or a biologically-active portion thereof as compared to the known compound.

In another embodiment, an assay is a cell-based assay comprising contacting a cell expressing a membrane-bound form of CML protein, or a biologically-active portion thereof, on the cell surface with a test compound and determining the ability of the test compound to modulate (e.g., stimulate or inhibit) the activity of the CML protein or biologically-active portion thereof. Determining the ability of the test compound to modulate the activity of a CML protein or a biologically-active portion thereof can be accomplished, for example, by determining the ability of the protein to bind to or interact with a CML protein target molecule. As used herein, a “target molecule” is a molecule with which CML protein binds or interacts in nature, for example, a molecule on the surface of a cell which expresses a CML protein interacting protein, a molecule on the surface of a second cell, a molecule in the extracellular milieu, a molecule associated with the internal surface of a cell membrane or a cytoplasmic molecule. A CML protein target molecule can be a non-CML molecule or a CML protein or polypeptide of the invention. In one embodiment, a CML protein target molecule is a component of a signal transduction pathway that facilitates transduction of an extracellular signal (e.g., a signal generated by binding of a compound to a membrane-bound CML protein molecule) through the cell membrane and into the cell. The target, for example, can be a second intercellular protein that has catalytic activity or a protein that facilitates the association of downstream signaling molecules with CML protein.

Determining the ability of the CML protein to bind to or interact with a CML protein target molecule can be accomplished by one of the methods described above for determining direct binding. In one embodiment, determining the ability of the CML protein to bind to or interact with a CML protein target molecule can be accomplished by determining the activity of the target molecule. For example, the activity of the target molecule can be determined by detecting induction of a cellular second messenger of the target (i.e., intracellular Ca²⁺, diacylglycerol, IP₃, etc.), detecting catalytic/enzymatic activity of the target an appropriate substrate, detecting the induction of a reporter gene (comprising a CML protein-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting CML protein or biologically-active portion thereof with a test compound and determining the ability of the test compound to bind to the CML protein or biologically-active portion thereof. Binding of the test compound to the CML protein can be determined either directly or indirectly as described above. In one such embodiment, the assay comprises contacting the CML protein or biologically-active portion thereof with a known compound which binds the protein or portion to form an assay mixture, contacting the assay mixture with a test compound, and determining the ability of the test compound to interact with a CML protein, wherein determining the ability of the test compound to interact with the protein comprises determining the ability of the test compound to preferentially bind to CML protein or biologically-active portion thereof as compared to the known compound.

In still another embodiment, an assay is a cell-free assay comprising contacting CML protein or biologically-active portion thereof with a test compound and determining the ability of the test compound to modulate (e.g., stimulate or inhibit) the activity of the CML protein or biologically-active portion thereof. Determining the ability of the test compound to modulate the activity of CML protein can be accomplished, for example, by determining the ability of the protein to bind to a CML protein target molecule by one of the methods described above for determining direct binding. In an alternative embodiment, determining the ability of the test compound to modulate the activity of CML protein can be accomplished by determining the ability of the protein to further modulate a CML protein target molecule. For example, the catalytic/enzymatic activity of the target molecule on an appropriate substrate can be determined as described, above.

In yet another embodiment, the cell-free assay comprises contacting the CML protein or biologically-active portion thereof with a known compound which binds CML protein to form an assay mixture, contacting the assay mixture with a test compound, and determining the ability of the test compound to interact with CML protein, wherein determining the ability of the test compound to interact with the protein comprises determining the ability of the CML protein to preferentially bind to or modulate the activity of a CML protein target molecule.

The cell-free assays of the invention are amenable to use of both the soluble form or the membrane-bound form of CML protein. In the case of cell-free assays comprising the membrane-bound form of the protein, it may be desirable to utilize a solubilizing agent such that the membrane-bound form of CML protein is maintained in solution. Examples of such solubilizing agents include non-ionic detergents such as n-octylglucoside, n-dodecylglucoside, n-dodecyhnaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either CML protein or its target molecule to facilitate separation of complexed from non-complexed forms of one or both of the proteins, as well as to accommodate automation of the assay. Binding of a test compound to CML protein, or interaction of CML protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-CML fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or CML protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, above. Alternatively, the complexes can be dissociated from the matrix, and the level of CML protein binding or activity determined using standard techniques.

Other techniques for immobilizing proteins on matrices can also be used in the screening assays of the invention. For example, either the CML protein or its target molecule can be immobilized utilizing conjugation of biotin and streptavidin. Biotinylated CML protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with CML protein or target molecules, but which do not interfere with binding of the CML protein to its target molecule, can be derivatized to the wells of the plate, and unbound target or CML protein trapped in the wells by antibody conjugation. Methods for detecting such complexes, in addition to those described above for the GST-immobilized complexes, include immunodetection of complexes using antibodies reactive with the CML protein or target molecule, as well as enzyme-linked assays that rely on detecting an enzymatic activity associated with the CML protein or target molecule.

In another embodiment, modulators of CML protein expression are identified in a method wherein a cell is contacted with a candidate compound and the expression of CML protein mRNA or protein in the cell is determined. The level of expression of CML mRNA or protein in the presence of the candidate compound is compared to the level of expression of CML mRNA or protein in the absence of the candidate compound. The candidate compound can then be identified as a modulator of CML mRNA or protein expression based upon this comparison. For example, when expression of CML mRNA or protein is greater (i.e., statistically significantly greater) in the presence of the candidate compound than in its absence, the candidate compound is identified as a stimulator of CML protein mRNA or protein expression. Alternatively, when expression of the mRNA or protein is less (statistically significantly less) in the presence of the candidate compound than in its absence, the candidate compound is identified as an inhibitor of CML mRNA or protein expression. The level of CML mRNA or protein expression in the cells can be determined by methods described herein for detecting CML mRNA or protein.

In yet another aspect of the invention, CML protein can be used as a “bait protein” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al, 1993. Biotechniques 14: 920-924; Iwabuchi, et al., 1993. Oncogene 8: 1693-1696; and Brent WO 94/10300), to identify other proteins that bind to or interact with CML protein (“CML protein-binding proteins” or “CML protein-bp”) and modulate its activity, Such CML protein-binding binding proteins are also likely to be involved in the propagation of signals by CML protein as, for example, upstream or downstream elements of the CML protein pathway.

The two-hybrid system is based on the modular nature of most transcription factors, which consist of separable DNA-binding and activation domains. Briefly, the assay utilizes two different DNA constructs. In one construct, the gene that codes for CML protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a CML protein-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with CML protein.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of a CML nucleic acid sequence, e.g., a portion or fragment of SEQ ID NO: 1 and/or 3, or fragments or derivatives thereof, can be used to map the location of the CML gene on a chromosome. The mapping of the CML sequence to chromosomes is an important first step in correlating this sequence with genes associated with disease.

Briefly, a CML gene can be mapped to chromosomes by preparing PCR primers (preferably 15-25 bp in length) from the CML sequence. Computer analysis of the CML sequence can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the CML nucleic acid sequence will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the CML sequence to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Additionally, differences in the DNA sequences between individuals affected and unaffected with a disease associated with the CML gene, e.g., malignancies such as leukemia and/or solid tumors, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The CML nucleic acid sequence of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” as described in U.S. Pat. No. 5,272,057).

Furthermore, the sequences of the invention can be used to provide an alternative technique that determines the actual base-by-base DNA sequence of selected portions of an individual's genome. Thus, the CML nucleic acid sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The CML nucleic acid sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences can comfortably provide positive individual identification with a panel of perhaps 10 to 1,000 primers that each yield a non-coding amplified sequence of 100 bases. If predicted CML protein coding sequences, e.g., SEQ ID NO: 1 and/or 3, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining CML protein and/or nucleic acid expression as well as CML protein activity, in the context of a biological sample (e.g., blood, serum, cells, tissue) to thereby determine whether an individual is afflicted with a disease or disorder, or is at risk of developing a disorder, associated with aberrant CML protein expression or activity. The invention also provides for prognostic (or predictive) assays for determining whether an individual is at risk of developing cancer. For example, mutations in a CML gene can be assayed in a biological sample. Such assays can be used for prognostic or predictive purpose to thereby prophylactically treat an individual prior to the onset of a disorder characterized by or associated with CML protein, nucleic acid expression or activity.

Another aspect of the invention provides methods for determining CML nucleic acid expression or CML protein activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of CML protein in clinical trials.

These and other agents are described in further detail in the following sections

Diagnostic Assays

An exemplary method for detecting the presence or absence of CML protein in a biological sample involves obtaining a biological sample from a test subject and contacting the biological sample with a compound or an agent capable of detecting CML protein or nucleic acid (e.g., mRNA, genomic DNA) that encodes CML protein such that the presence of CML protein or nucleic acid is detected in the biological sample. An agent for detecting CML mRNA or genomic DNA is a labeled nucleic acid probe capable of hybridizing to CML mRNA or genomic DNA. The nucleic acid probe can be, for example, a full-length CML nucleic acid, or a portion thereof, such as an oligonucleotide of at least 15, 30, 50, 100, 250 or 500 nucleotides in length and sufficient to specifically hybridize under stringent conditions to CML mRNA or genomic DNA. Other suitable probes for use in the diagnostic assays of the invention are described herein.

An agent for detecting CML protein is an antibody capable of binding to CML protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect CML mRNA, protein, or genomic DNA in a biological sample in vitro as well as in vivo. For example, in vitro techniques for detection of CML mRNA include Northern hybridizations and in situ hybridizations. In vitro techniques for detection of CML protein include enzyme linked immunosorbent assays (ELISAs), Western blots, immunoprecipitations, and immunofluorescence. In vitro techniques for detection of CML genomic DNA include Southern hybridizations. Furthermore, in vivo techniques for detection of CML protein include introducing into a subject a labeled anti-CML protein antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting CML protein, mRNA, or genomic DNA, such that the presence of CML protein, mRNA or genomic DNA is detected in the biological sample, and comparing the presence of CML protein, mRNA or genomic DNA in the control sample with the presence of CML protein, mRNA or genomic DNA in the test sample.

The invention also encompasses kits for detecting the presence of CML protein in a biological sample. For example, the kit can comprise: a labeled compound or agent capable of detecting CML protein or mRNA in a biological sample; means for determining the amount of CML protein or mRNA in the sample; and means for comparing the amount of CML protein in the sample with a standard. The compound or agent can be packaged in a suitable container. The kit can further comprise instructions for using the kit to detect CML protein or nucleic acid.

Prognostic Assays

The diagnostic methods described herein can furthermore be utilized to identify subjects having or at risk of developing cancer. For example, the assays described herein, such as the preceding diagnostic assays or the following assays, can be utilized to identify a subject having or at risk of developing. Alternatively, the prognostic assays can be utilized to identify a subject having or at risk for developing a disease or disorder. Thus, the invention provides a method for identifying a disease or disorder associated with aberrant CML protein expression or activity in which a test sample is obtained from a subject and CML protein or nucleic acid (e.g., mRNA, genomic DNA) is detected, wherein the presence of CML protein or nucleic acid is diagnostic for a subject having or at risk of developing a disease or disorder associated with aberrant CML protein expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat the cancer. Thus, the invention provides methods for determining whether a subject can be effectively treated with an agent for a cancer associated with CML protein expression or activity in which a test sample is obtained and CML protein or nucleic acid is detected (e.g., wherein the presence of CML protein or nucleic acid is diagnostic for a subject that can be administered the agent to treat a disorder associated with aberrant CML protein expression or activity).

The methods of the invention can also be used to detect genetic lesions in a CML gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding CML protein, or the mis-expression of the CML gene. For example, such genetic lesions can be detected by ascertaining the existence of at least one of: (i) a deletion of one or more nucleotides from a CML gene; (ii) an addition of one or more nucleotides to a CML gene; (iii) a substitution of one or more nucleotides of a CML gene, (iv) a chromosomal rearrangement of a CML gene; (v) an alteration in the level of a messenger RNA transcript of a CML gene, (vi) aberrant modification of a CML gene, such as of the methylation pattern of the genomic DNA, (vii) the presence of a non-wild-type splicing pattern of a messenger RNA transcript of a CML gene, (viii) a non-wild-type level of a CML protein, (ix) allelic loss of a CML gene, and (x) inappropriate post-translational modification of a CML protein. As described herein, there are a large number of assay techniques known in the art which can be used for detecting lesions in a CML protein gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the CML protein gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to the CML gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a CML gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in CML can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in CML sequences can be identified in two dimensional arrays containing light-generated DNA probes as described in Cronin, et al., above. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the CML gene and detect mutations by comparing the sequence of the sample CML gene with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the CML protein gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type CML sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in CML cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a CML nucleic acid sequence, e.g., a wild-type CML sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in a CML gene. For example, single-strand conformation polymorphism (SSP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition, it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a CML protein gene.

Furthermore, any cell type or tissue, preferably liver cells, in which CML protein is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on CML protein activity (e.g., CML protein gene expression), as identified by a screening assay described herein can be administered to individuals to treat (prophylactically or therapeutically) disorders (e.g., malignancies such as leukemia and/or solid tumors) associated with aberrant CML protein activity.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of CML protein, expression of CML nucleic acid, or mutation content of CML protein genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so-called ultra-rapid metabolizers who do not respond to standard doses. Recently, the molecular basis of ultra-rapid metabolism has been identified to be due to CYP2D6 gene amplification.

Thus, the activity of CML protein, expression of CML protein nucleic acid, or mutation content of CML genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when appplied to dosing or drug selection, can avoid adverse reactions or therapeutic failure and thus enhance therapeutic or prophylactic efficiency when treating a subject with a CML protein modulator, such as a modulator identified by one of the exemplary screening assays described herein.

Methods of Treatment

The invention provides for both prophylactic and therapeutic methods of treating a subject at risk of (or susceptible to) a cancer associated with CML protein expression or activity. These methods of treatment will be discussed more fully below.

Another aspect of the invention pertains to methods of modulating CML protein expression or activity for therapeutic purposes. The modulatory method of the invention involves contacting a cell with an agent that modulates one or more of the activities of CML protein activity associated with the cell. An agent that modulates CML protein activity can be an agent as described herein, or a nucleic acid or a protein, a naturally-occuring cognate ligand of CML protein, a peptide, a CML protein peptidomimetic, or other small molecule. In one embodiment, the agent stimulates one or more CML protein activity. Examples of such stimulatory agents include active CML protein and a nucleic acid molecule encoding CML protein that has been introduced into the cell. In another embodiment, the agent inhibits one or more CML protein activities. Examples of such inhibitory agents include antisense CML nucleic acid molecules and anti-CML protein antibodies. These modulatory methods can be performed in vitro (e.g., by culturing the cell with the agent) or, alternatively, in vivo (e.g., by administering the agent to a subject). As such, the invention provides methods of treating an malignancy-related disease or disorder in a subject, e.g., a mammal, characterized by aberrant expression or activity of CML protein or nucleic acid molecule, e.g., malignant cell growth. In one embodiment, the method involves administering an agent (e.g., an agent identified by a screening assay described herein), or combination of agents that modulates (e.g., up-regulates or down-regulates) CML protein expression or activity. In another embodiment, the method involves administering CML protein or nucleic acid molecule as therapy to compensate for reduced or aberrant CML protein expression or activity. As described above, CML protein activity or expression may be modulated as therapy for, e.g., malignant cell growth.

Both the novel nucleic acids encoding CML protein, and the CML protein of the invention, or fragments thereof, may also be useful in diagnostic applications, wherein the presence or amount of the nucleic acid or the protein are to be assessed. These materials are further useful in the generation of antibodies that immunospecifically-bind to the novel substances of the invention for use in therapeutic or diagnostic methods.

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

The invention will be further illustrated in the following non-limiting examples.

### EXAMPLES

### Example 1

### Expression of CML28 in Tumor Cell Lines and Normal Tissues

A CML cDNA library construction and screening methods are described in the art [9]. Briefly, mRNA was extracted from peripheral blood mononuclear cells (PBMC) from 3 patients with CML, one with accelerated phase and two with stable phase disease using standard methods and pooled to create a representational CML expression library in a λ bacteriophage expression vector. Filters with recombinant phage were then incubated with post-DLI patient serum, diluted at 1:500 and alkaline phosphatase-conjugated anti-human IgG. A human testis cDNA library (1×10⁶ phage) derived from normal whole human testes pooled from 11 males (Clontech, Palo Alto Calif.) was screened with a 0.5 kb ³²P-labeled CML28 cDNA probe, as previously described [15]. After three rounds of phage plaque purification, five positive clones were identified, converted into plasmid pTriplEx by cre-lox-mediated excision, and sequenced in both strands. Specifically, a novel 0.9 kb cDNA clone from a CML λ expression cDNA library was identified. This clone was identified because of reactivity with sera obtained from a patient with CML who developed an effective immune response to their leukemia after donor lymphocyte infusion. The 0.9 kb clone contained a 700 bp incomplete open reading frame (ORF) with 5′ end missing and had no significant sequence homology to any known genes in GenBank or other databases by using a BLAST program (NCBI, NIH).

Serum was obtained at various time points before and after donor lymphocyte infusion in patients enrolled on a clinical trial of CD4+ DLI for treatment of relapse after allogeneic BMT ([6]). Serum samples were also obtained from patients with metastatic melanoma or metastatic non-small cell lung carcinoma upon enrollment into IRB approved tumor cell vaccine trials [14]. Serum samples were obtained from patients with prostate cancer enrolled in the genitourinary clinic at DFCI.

Multiple tissue Northern blots were prepared with purified polyA+RNAs (Clontech human cancer cell line blot, human normal tissue I blot, human normal tissue II blot, and human normal 12-lane blot, Palo Alto Calif.). Hybridizations were conducted with a 0.5 kb ³²P-labeled CML28 probe in the ExpressHyb™ hybridization solution (Clontech, Palo Alto Calif.) at 68° C. for one hour according to the manufacturer's protocol. The same blots were then stripped and hybridized with the ³²P-labeled human β-actin cDNA probe (Clontech, Palo Alto Calif.) as controls.

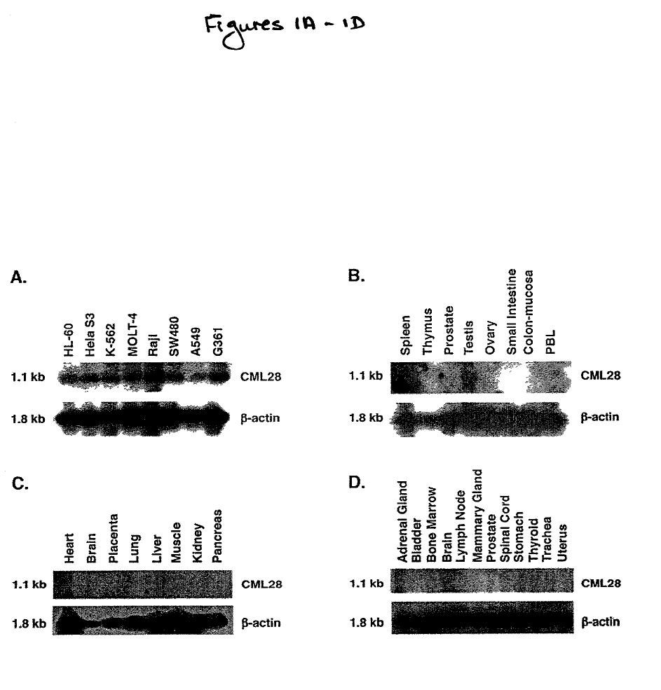

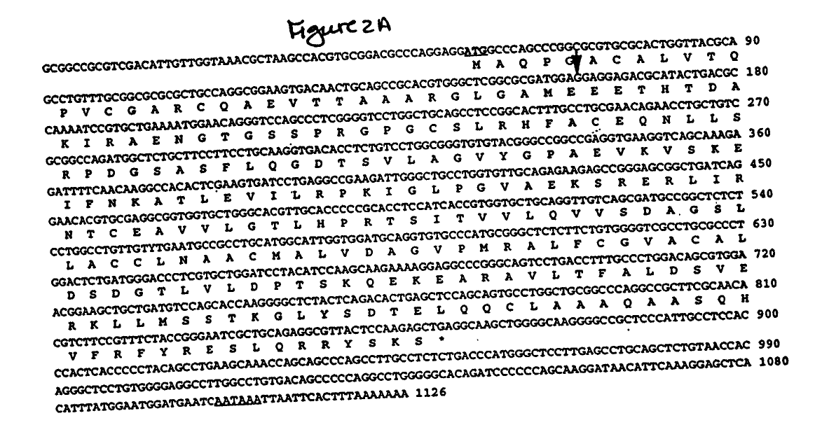

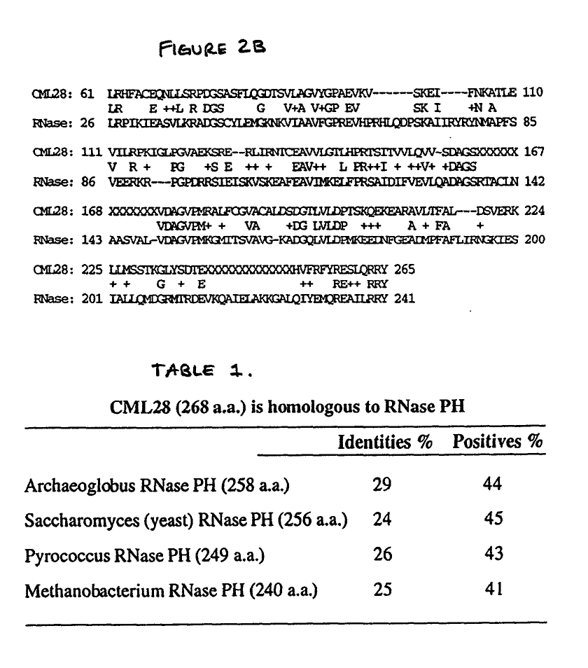

As shown in FIG. 1A, Northern blot hybridizations with this cDNA probe showed that this gene had a 1.1 kb transcript and was highly expressed in each of 8 human tumor cell lines that was examined. Northern blots with 2 μg polyA+mRNA were obtained from eight tumor cell lines and 28 normal tissues was hybridized with a CML28 cDNA probe. The size of the transcript is indicated on the left of the FIG. 1. β-actin mRNA loading controls for each lane on the same blots were revealed by hybridization with a human β-actin cDNA probe.

These included HL-60, K562, Molt-4 and Raji cell lines derived from myeloid or lymphoid tumors as well as 4 cell lines derived from a variety of epithelial malignancies and melanoma. In contrast, Northern blots only revealed expression of CML28 in 1 of 26 normal human tissues (FIGS. 1B, 1C and 1D), in human testis. Although background binding was noted in spleen, no specific hybridization band was noted in this tissue.

### Example 2

### Cloning of CML28 cDNA

A normal human testis cDNA library was screened to clone the normal CML28 gene. These experiments identified a 1.126 kb sequence which contains 55 bp of 5′untranslated region (UTR), a 804 bp open reading frame (ORF) with 268 amino acids and a 264 bp 3′UTR. The DNA sequence at the start codon in the ORF contained a Kozak consensus sequence for protein translation in a high efficiency [16]. In vitro transcription and translation assay (TNT) confirmed that this long ORF was functional and that it encoded a 28 kD protein, which correlated well with the predicted molecular weight. A polyadenylation signal was found in the 3′ UTR, suggesting that this transcript had a complete 3′ UTR sequence upstream of the poly A tail. This correlated well with the 1.1 kb size of the gene shown in the Northern blot (FIG. 1A). Since this antigen was 28 kD in size and was originally isolated from a CML library, it was termed CML28. CML28 cDNA sequence has been submitted to GenBank (accession number: AF285785).

Comparison of the normal sequence of this gene isolated from the testis library with the sequence isolated from the CML library demonstrated that two cDNA sequences were identical in the 0.9 kb 3′overlapping region, suggesting that immunogenicity of this antigen may not be resulted from mutations.

Completion of CML28 ORF enabled us to search potential homologous in protein databases. By using the Clusters of Orthologous Groups of proteins program rather than a BLAST program (NCBI, NIH), CML28 showed a 45% homology (24-29% identities) spreading all over its ORF to bacterial (258 a.a.) and yeast RNase PH (256 a.a.) (FIG. 2B), suggesting that CML28 may be a human homologous of RNase PH. DNA sequence analysis. Sequence homology searches were performed using the GenBank databases (NCBI, NIH). Other DNA and protein sequence and structure analysis were performed with either the GCG program (Genetics Computer Group, Madison, Wis.) or the Lasergene program (DNASTAR, Madison, Wis.).

### Example 3

### Human Genomic DNA Library Screening and FISH Chromosome Localization Analysis of CML 28.

1×10⁶ phage from a lambda Dash II human genomic DNA library (Stratagene, La Jolla Calif.) were screened using described methods [15]. Genomic DNAs from purified positive phage were prepared using Qiagen Lambda Midi Kit (Valencia, Calif.). The insert size of positive genomic DNA clones was determined by gel electrophoresis. Exon sequences in the genomic DNA clones encoding CML28 cDNA were confirmed by DNA sequencing.

Human FISH chromosomal localization was performed using a CML28 genomic clone with an insert of 18 kb labeled with digoxigenin dUTP by nick translation (Incyte Genomics, St. Louis, Mo.). Labeled probe was combined with sheared human DNA and hybridized to metaphase chromosomes derived from PHA stimulated peripheral blood lymphocytes in a solution containing 50% formamide, 10% dextran sulfate and 2×SSC. Specific hybridization signals were detected by incubating the hybridized slides with fluoresceinated anti-digoxigenin antibodies followed by counterstaining with DAPI.

Restriction enzyme analysis of normal human genomic DNA followed by Southern blot hybridization with CML28 cDNA probe suggested that CML28 was a single copy gene [17]. Screening of a λ human genomic DNA library (Stratagene, Calif.) with CML28 cDNA probe resulted in the identification of one clone with 18 kb insert.

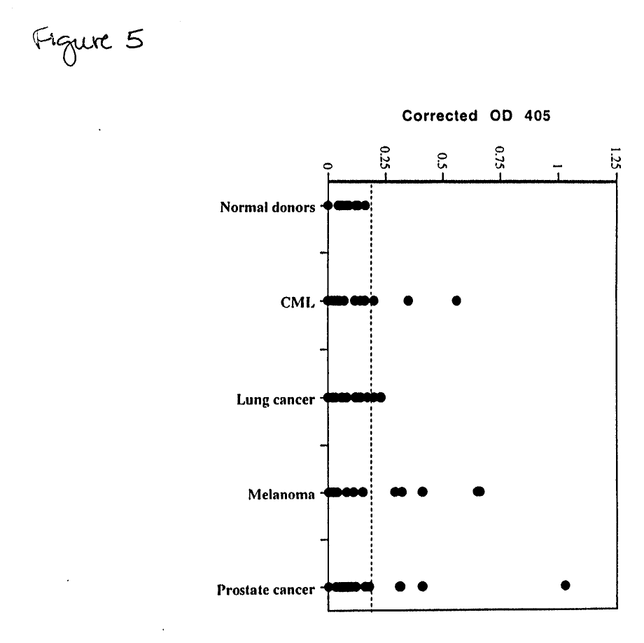

Human chromosomal localization of CML28 was performed by FISH using a 18 kb CML28 genomic DNA clone as a probe. A total of 80 metaphase cells were analyzed with 68 (85%) exhibiting specific labeling. Based on size, morphology and band pattern of specifically-labeled chromosome, CML28 was localized to chromosome 19. The further cohybridization with both CML28 clone and an anonymous genomic clone which has been previously mapped to 19p13 resulted in the specific labeling of the long and short arm of chromosome 19. Measurement of 10 specifically labeled chromosome 19 demonstrated that CML28 is located at a position which is 46% of the distance from the centromere to the telomere of chromosome arm 19q, an area which corresponds to band 19q13.13-13.2 (FIG. 3) Metaphase spreads of PBL stimulated with PHA were hybridized with a 18 kb CML28 genomic DNA probe. The CML28 specific hybridization signals are identified with arrows. The schematic representation of chromosome 19 on the right illustrates the chromosomal position of CML28 at 19q13. The chromosome 8 representation is from the International System for Human Cytogenetic Nomenclature 1995.

### Example 4

### Antibody Response to CML28

To define the immunogenicity of CML28 as a potential tumor antigen, GST-CML28 fusion protein was constructed and used to analyze antibody reactivity in normal and CML patient sera. A cDNA fragment encoding CML28 with EcoRi restriction site on both ends was generated by PCR using high-fidelity enzyme Pfu Turbo DNA polymerase (Stratagene, Calif.) and primers 59E (5′-CGGAGAATTCGGAGACGCATACTGACGCCAAAATC-3′; SEQ ID NO: 5) and 59 F (5′-CGGAGAATTCCCTCAGCTCTTGGAGTAACGCCT-3′; SEQ ID NO: 6). The underlined sequences in these primers were designed for subcloning into EcoRi site of GST fusion vector pGEX-3×(Amersham-Pharmacia, Piscataway, N.J.). CML28 fragment was fused in frame to the C-terminus of GST protein after cloning into the EcoRI site of the GST expression vector pGEX-3×and were further examined by DNA sequencing before transformation into the BL-21 strain of the E. coli. The GST and the fusion protein GST-CML28 were purified according to the manufacturer's protocols (Amersham-Pharmacia, N.J.) or with B-per Bacterial Protein Extraction Reagent (Pierce, Rockford, Ill.).

The purified GST-CML28 fusion protein has a molecular weight of 58 kD corresponding to the combined size of GST (30 kD) and CML28. Recombinant proteins expressed in transformed E. coli were subjected to 10-12% SDS-PAGE with Tris-Glycine buffer and transferred onto nitrocellulose filters in 20% methanol in Tris-Glycine buffer. Proteins on the blots were visualized as previously described [9]. Purified GST or GST-CML28 fusion protein was loaded onto separate lanes as indicated. After electrophoresis, the Western blots were probed with anti-GST antibody revealing a 58 kD band in all lanes containing GST-CML28 fusion protein (upper blots) and a 30 kD protein in all lanes containing GST only. This blot confirms the loading of either GST or GST-CML28 and also demonstrates the size of the GST-CML28 fusion protein and its reactivity with anti-GST. After stripping, this Western blot was divided into four equivalent parts and probed with normal donor sera as well as with patient sera collected at different times indicated in the lower part of the figure; pre-BMT, pre-DLI and post-DLI. The molecular size of GST-CML28 (58 kD) reacting with post-DLI serum is the same as that revealed with anti-GST. In the Western blots shown in FIG. 4, antibodies to CML28 were not detected in normal sera but were detected in sera obtained from a patient with CML 6 months after donor lymphocyte infusion (DLI). Serum from this patient had been used to screen the CML expression library and this result therefore confirmed that the CML28 protein had been immunogenic in vivo. Antibodies to GST-CML28 were not detected in serum from the same patient obtained prior to allogeneic bone marrow transplant or prior to DLI.

### Example 5A

### Quantitation of Specific IgG Response to CML28 in Normal Donors and Patients with Cancer

To further characterize the serological response to CML28, a sensitive ELISA assay to detect and quantitate the levels of specific IgG antibody in sera obtained from normal donors and patients with different malignancies was developed. Detection of CML28 specific antibody in patient sera by ELISA assay. ELISA plates (VWR Scientific, NJ) were coated with 50 μl of purified recombinant protein at 5 μg/ml in coating buffer (PBS+0.05% sodium azide) overnight at 4° C. [9]. Plates were washed with PBS with 0.05% Triton X-100, and blocked overnight at 4° C. with 200 μl/well of 2% nonfat milk with 0.05% Triton X-100. 50 μl/well patient sera was added to a final dilution of 1:1000, and incubated at room temperature for 3 hours. The procedure for detection of specific IgG antibody has been described previously [9].

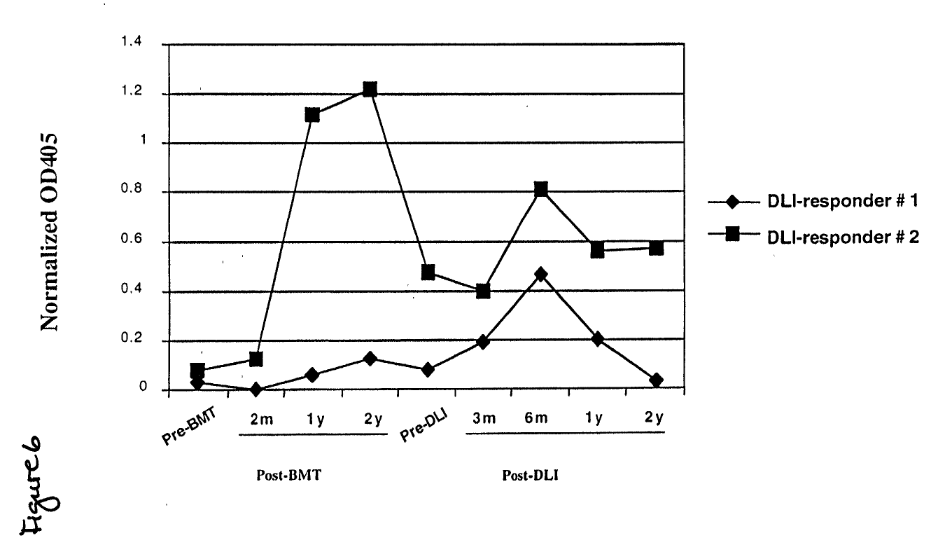

In this assay, antibody reactivity with purified GST-CML28 was compared to antibody reactivity with purified GST. Serum samples from normal donors (n=10), patients with CML (n=18), lung cancer (n=15), melanoma (n=17), and prostate cancer (n=15) were analyzed in each group at a dilution of 1:1000. Specific reactivity against CML28 in each sample presented as corrected OD405 was determined by subtracting the level of reactivity to GST alone. The line at 0.1793 represents the upper limit of background OD in normal donors (mean±2SD). As summarized in FIG. 5, reactivity was not detected in normal donors (n=10) but specific CML28 reactivity was detected in sera from patients with lung cancer (2 of 15 patients), melanoma (5 of 17 patients) and prostate cancer (5 of 15 patients). In 3 who had positive reactivity out of 18 patients with CML, the highest level of reactivity was observed in the patient known to have specific antibody by Western blot. In each instance where reactivity against GST-CML28 was greater than reactivity against GST, ELISA reactivity was blocked by prior incubation of sera with excess purified GST-CML28. These results confirm the specificity of the response to CML28 in these patients and suggested that CML28 was capable of eliciting humoral immune responses in patients with a variety of tumors.

The immune response to CML28 was further examined in the patient with CML who had been found to have high titer antibody 1 year after DLI. The specific CML28 ELISA was used to measure the antibody response to this antigen in serial serum samples obtained prior to transplant and at various times over a 2 year period after DLI. Quantitative assessment of CML28-specific IgG antibody was determined in serial serum samples from two patients with relapsed CML who responded to DLI. The X-axis indicates the time of the sampling. The Y-axis presents the specific OD values by ELISA. The percent marrow metaphases containing the Philadelphia chromosome as well as results of PCR analysis of patient blood and marrow samples for the presence of bcr-abl mRNA are also indicated.

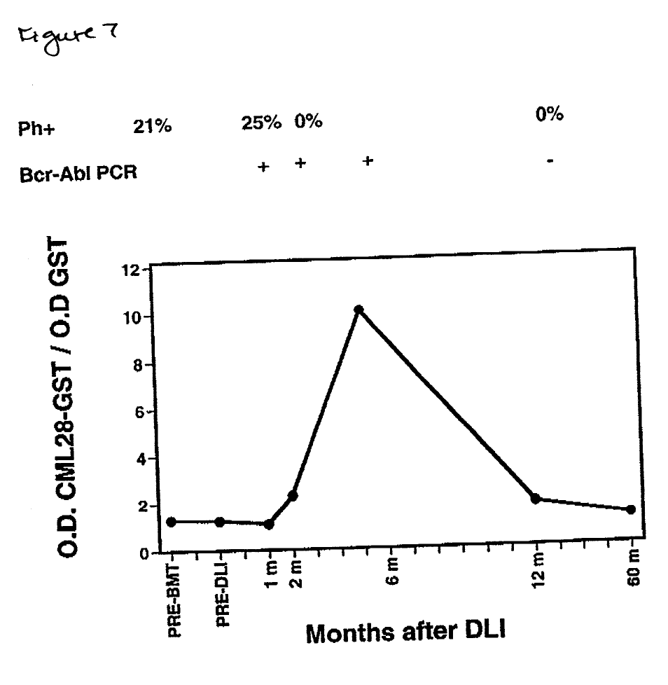

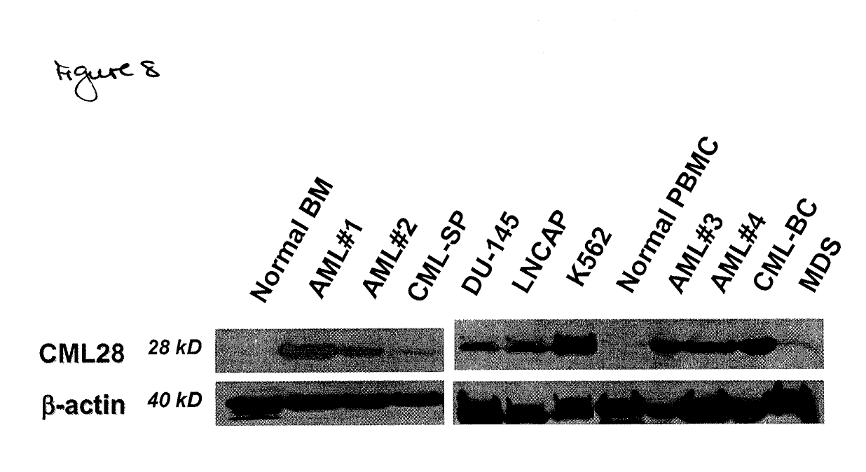

As shown in FIGS. 6 and 7, antibodies to CML28 were not detectable before BMT and before DLI in DLI-responder #1. Antibody titers to CML28 increased markedly 3 months post-DLI and persisted at high levels for 1 year. Specific antibody was no longer detectable 2 years after DLI. The time course of antibody reactivity in this patient correlated well with the onset of cytogenetic response. Despite achieving a complete cytogenetic remission at 3 months post-DLI, bcr-abl mRNA remained detectable in blood and bone marrow until a molecular remission was achieved 12 months post-DLI. In the second DLI responder, antibody to CML28 increased markedly 1 year after BMT and persisted at high levels for another year. This antibody reactivity to CML28 was dropped in relapse of CML. Furthermore, 6 months after DLI the antibody reactivity to CML28 in the second patient increased again and maintained in a high level at least for two years after DLI, which correlated well with the CML remission in this patient.

### Example 5B

### Determination of the Distribution of a CML28 Polypeptide in Hematopoietic Tissues, Cell Lines and Primary Leukemias Using an Anti-CML28 Murine Monoclonal Antibody.

Protein expression of CML28 was also examined in primary normal and malignant hematopoietic tissue samples by western blotting using a monoclonal antibody specific for CML28. A representative series of samples tested is shown in FIG. 8. CML28 was found in high levels a variety of cell lines including K562, BV173, Jurkat, and prostate cell lines DU-145 and LNCAP. CML28 protein levels were consistently low or undetectable in lysates prepared from the 3 normal bone marrow and 4 normal G-CSF stimulated peripheral blood mononuclear cell samples analyzed. In contrast, CML28 was found in high levels in 8 of 9 samples tested from patients with acute myeloid leukemia. CML28 was present at low levels or not detectable by western blot in cell lysates from 2 myelodysplasia and 8 stable phase CML samples tested. Of all the primary leukemia samples tested, CML28 levels were highest in the 4 CML blast crisis samples.

### Example 6

### Expression of CML66 in Tumor Cell Lines and Normal Tissues

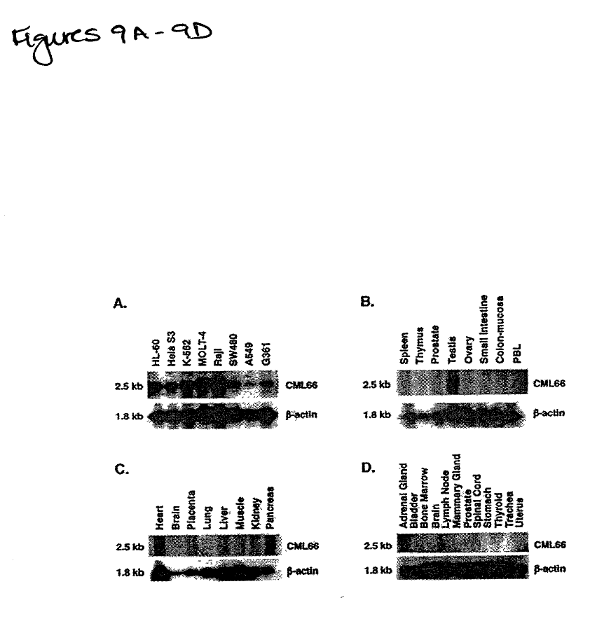

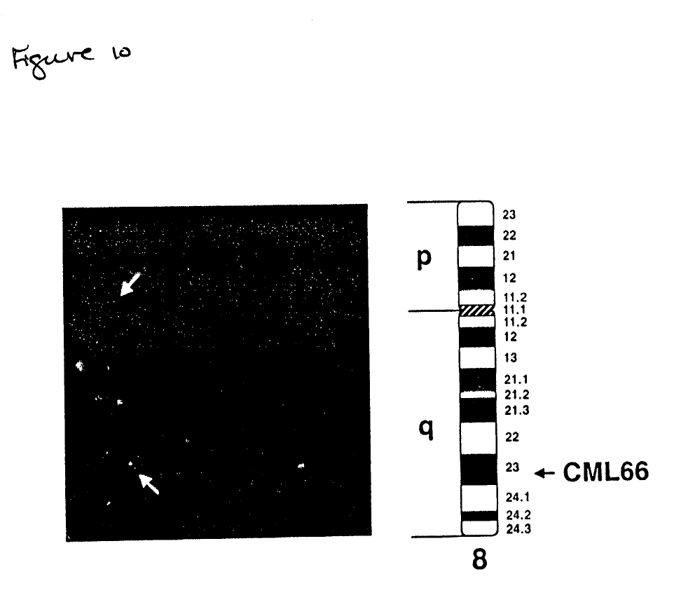

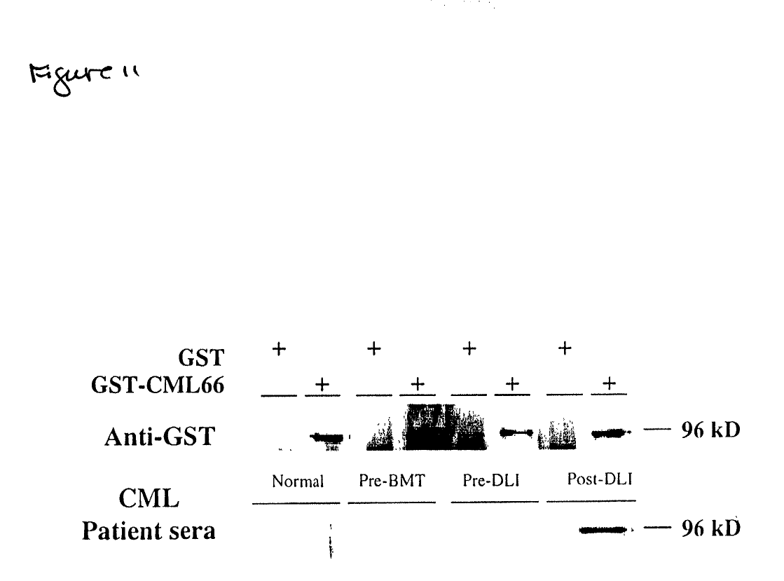

A novel 2.1 kb cDNA clone from a CML λ expression cDNA library was identified (16). This clone was identified because of reactivity with sera obtained from a patient with CML who developed an effective immune response resulting in complete remission of leukemia after donor lymphocyte infusion. The 2.1 kb clone had no significant sequence homology to any known genes in GenBank or other databases (NCBI, NIH). As shown in FIG. 9A, Northern blot hybridizations with this cDNA probe showed that this gene had a 2.5 kb transcript and was highly expressed in 7 of 8 human tumor cell lines that were examined. These included HL-60, K562, Molt-4 and Raji cell lines derived from myeloid or lymphoid tumors as well as 4 cell lines derived from a variety of epithelial malignancies and melanoma. In contrast, Northern blots only revealed expression of CML66 in 2 of 26 normal human tissues (FIGS. 9B-9D). As shown in FIGS. 9B-9D, CML66 was expressed at relatively high levels in human testis and at lower levels in heart. Although background binding was noted in pancreas, no specific hybridization band was noted in this tissue.

Multiple tissue Northern blots were prepared with purified polyA+RNAs (Clontech human cancer cell line blot, human normal tissue I blot, human normal tissue II blot, and human normal 12-lane blot, Palo Alto Calif.). Hybridizations were conducted with a 0.8 kb ³²P-labeled CML66 probe in the ExpressHyb™ hybridization solution (Clontech) at 68° C. for one hour according to the manufacturer's protocol. The same blots were then stripped and hybridized with the ³²P-labeled human β-actin cDNA probe (Clontech) as controls.

### Example 7

### Cloning of CML66 cDNA

A normal human testis cDNA library was screened to clone the normal CML66 gene. The entire cDNA sequence was completed using 5′RACE. These experiments identified a 2,319 bp sequence which contains 242 bp of 5′untranslated region (UTR), a 1749 bp open reading frame (ORF) with 583 amino acids and a 338 bp 3′UTR (based on computer analysis). The DNA sequence at the start codon in the ORF contained a Kozak consensus sequence for high efficiency protein translation (21). In vitro transcription and translation confirmed that this long ORF encoded a 66 kD protein. A polyadenylation signal was found in the 3′ UTR. In addition, 5′ end primer extension experiments (Promega, Madison, Wis.) indicated that the transcription starting site was located 200 bp upstream of the 5′end of this cloned transcript. This correlated well with the 2.5 kb size of the gene shown in Northern blots. Since this antigen was 66 kD in size and was originally isolated from a CML library, it was termed CML66. CML66 cDNA sequence has been submitted to GenBank (accession number: AF283301).

CML cDNA library construction and screening were previously described (16). Briefly, mRNA was extracted from peripheral blood mononuclear cells (PBMC) from 3 patients with CML using standard methods and pooled to create a representational CML expression library in a λ bacteriophage expression vector. Filters with recombinant phage were then incubated with post-DLI patient serum (1:500 dilution) and alkaline phosphatase-conjugated anti-human IgG.

Serum was obtained at various times before and after lymphocyte infusion in patients enrolled on a clinical trial of CD4+DLI for treatment of relapse after allogeneic BMT (15). Serum samples were also obtained from patients with metastatic melanoma or metastatic non-small cell lung carcinoma upon enrollment into IRB approved tumor cell vaccine trials (19). Serum samples were obtained from patients with hormone refractory advanced prostate cancer at the Dana-Farber Cancer Institute.

A human testis cDNA library (1×10⁶ phage) derived from normal whole human testes pooled from 11 males (Clontech, Palo Alto Calif.) was screened with a 0.8 kb ³²P-labeled CML66 probe, as previously described (20). After three rounds of phage plaque purification, 5 positive clones were identified, converted into plasmid pTriplEx by cre-lox-mediated excision, and sequenced in both strands.

Total RNA was prepared from cultured tumor cell lines, patient CML cells, and normal human PBMC using RNAzole (Tel-Test, Friendswood, Tex.). RT-PCR and PCR cloning were performed as described previously (20). A sense primer (25 k) specific for the 5′-upstream CML66 (5′-CGGAGAATTCGGCACGAGTCCCAGTCTCTGTGCGA-3′; SEQ ID NO: 7), and a second antisense primer (25c) specific for the 3′-downstream CML66 (5′-CGGAGAATTCTCATTCTCTGTATTTACTTTTATTAA-3′; SEQ ID NO: 8) were used for PCR cloning. All of the PCR cloning reactions were performed using high fidelity enzymes such as Pfu Turbo (Stratagene). The 5′ rapid amplification of cDNA ends (5′RACE) by PCR was performed using the human testis Marathon-Ready™ cDNAs as templates with a CML66 specific antisense primer 25H (5′-CCCAGGTAGAAGATGAGAAATGGATA-3′; SEQ ID NO: 9) and the primer AP1 or AP2 specific for the adapter sequence (Clontech). PCR-amplified products were subdloned into the pCRII-TOPO vector (Invitrogen, Carlsbad, Calif.), followed by DNA sequencing.

### Example 8

### cDNA Sequence Comparison of CML66 Gene in Normal Tissues and Tumor Cells

CML66 cDNA was cloned by screening a normal human testis cDNA library using CML66 cDNA isolated from the CML library as a probe. Five separate clones of different lengths were sequenced in both strands and all overlapping regions were found to have identical sequence. Comparison of the normal CML66 gene isolated from the testis library with the sequence isolated from the CML library demonstrated that the cDNA sequences were identical except for two single nucleotide changes (FIG. 16). One substitution at bp 1412 resulted in a change from Asn to His at amino acid 394. A second substitution at bp 1509 resulted in a change from Asn to Ser at amino acid 426. Three cDNA clones of different lengths isolated from the CML library were sequenced in both strands, and all contained these two single nucleotide differences.

CML66 cDNA was amplified by high fidelity PCR from leukemia cells from 3 additional patients with CML, one patient with acute myelogenous leukemia (AML) and a panel of tumor cell lines. CML cells from one of these patients (CML-P1) had been used to construct the CML cDNA library and the CML66 sequence in this individual was identical to the CML library sequence. DNA sequence of 9 CML66 clones from these tumor cells was compared to the sequence derived from normal testis and 17 additional single bp mutations were identified (FIG. 16). Three mutations were silent but 14 mutations resulted in amino acid substitutions. None of these mutations resulted in premature stop codons or reading frame shifts and 2 mutations (F252L and V269I) occurred in multiple tumor cells.

### Example 9

### Chromosome Localization of CML66

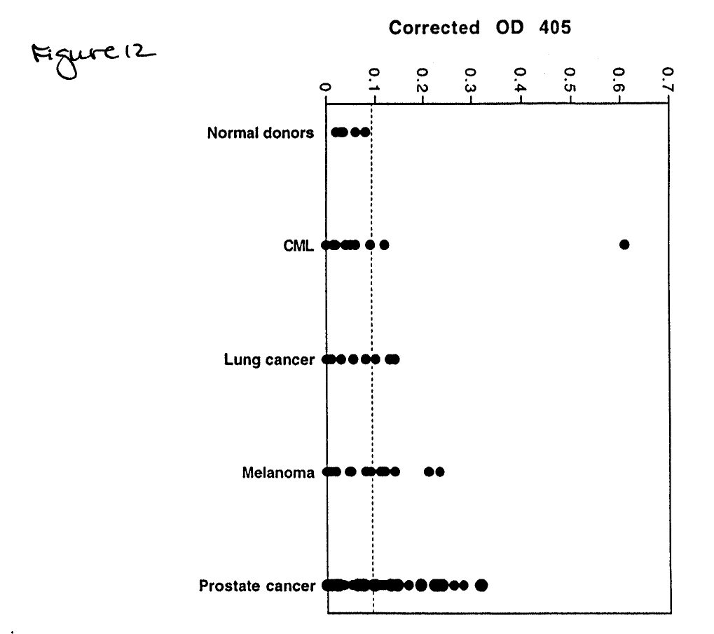

Restriction enzyme analysis of normal human genomic DNA followed by Southern blot hybridization with CML66 cDNA probe suggested that CML66 was a single copy gene (22). Human chromosome localization of CML66 was performed by FISH using a 23 kb CML66 genomic DNA clone as a probe. A total of 80 metaphase cells were analyzed with 62 (78%) exhibiting specific labeling. Based on size, morphology and band pattern of specifically-labeled chromosomes, CML66 was localized to chromosome 8. Co-hybridization with both CML66 clone and an anonymous genomic clone which had been previously mapped to 8q12 resulted in labeling of the long arm of chromosome 8 at two distinct loci. Measurement of 10 specifically labeled chromosome 8 demonstrated that CML66 is located at a position which is 67% of the distance from the centromere to the telomere of chromosome arm 8q, an area which corresponds to band 8q23.3 (FIG. 10) (23).

1×10⁶ phage from a lambda Dash II human genomic DNA library (Stratagene, La Jolla Calif.) were screened using described methods (20). Genomic DNAs from purified positive phage were prepared using Qiagen Lambda Midi Kit (Qiagen, Valencia, Calif.). The insert size of positive genomic DNA clones was determined by gel electrophoresis. Exon sequences in the genomic DNA clones encoding CML66 cDNA were confirmed by DNA sequencing.

Human FISH chromosomal localization was performed using a CML66 genomic clone with an insert of 23 kb labeled with digoxigenin dUTP by nick translation (Incyte Genomics, St. Louis Mo.). Labeled probe was combined with sheared human DNA and hybridized to metaphase chromosomes derived from PHA stimulated peripheral blood lymphocytes in a solution containing 50% formamide, 10% dextran sulfate and 2×SSC. Specific hybridization signals were detected by incubating the hybridized slides with fluorescein-conjugated anti-digoxigenin antibodies followed by counterstaining with DAPI.

### Example 10

### Antibody Response to CML66 After Allogeneic BMT and DLI

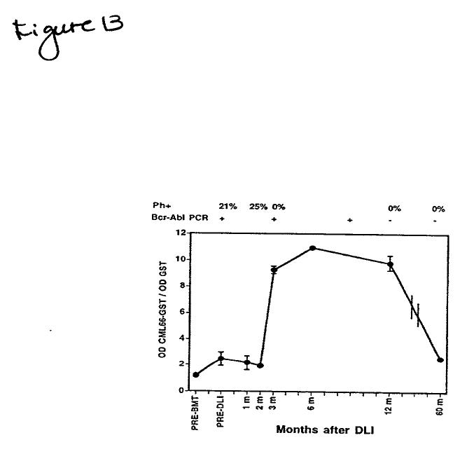

To characterize the immunogenicity of CML66 as a tumor rejection antigen, GST-CML66 fusion protein was purified and used as a probe to analyze antibody reactivity in normal and CML patient sera. The purified GST-CML66 fusion protein has a molecular weight of 96 kD corresponding to the combined size of GST (30 kD) plus the complete ORF of CML66. In the Western blots shown in FIG. 11, antibodies to CML66 were not detected in normal sera but were detected in sera obtained from a patient with CML 1 year after DLI. Serum from this patient had been used to screen the CML library and this result therefore confirmed that the CML66 protein had been immunogenic in vivo. In these Western blots, antibodies to GST-CML66 were not detected in serum from the same patient obtained prior to allogeneic BMT or prior to DLI.

To provide a more sensitive method for detecting and quantifying the immune response to CML66 an ELISA using purified GST-CML66 was developed. As shown in FIG. 13, IgG antibodies to CML66 were also not detectable by ELISA before BMT and before DLI. Antibody titers to CML66 increased markedly 3 months post-DLI and persisted at high levels for at least 1 year. Specific antibody was no longer detectable 5 years after DLI. The time course of antibody reactivity in this patient correlated well with the onset of cytogenetic response. After achieving a complete cytogenetic remission 3 months post-DLI, bcr-abl mRNA remained detectable in blood and bone marrow until a molecular remission was achieved 12 months post-DLI. Further characterization of the antibodies reacting with CML66 demonstrated that they were primarily IgG1 and IgG4 isotypes.

A cDNA fragment encoding full-length long ORF (ORF 1) of CML66 with EcoRI restriction site on both ends was generated by PCR using high-fidelity enzyme Pfu Turbo DNA polymerase (Stratagene) and primers 25F1 (5′-CGGAGAATTCGATGGAGGTGGCGGCTAATTGCTCC-3′; SEQ ID NO: 10) and 25c. The underlined sequences in these primers were designed for subcloning into EcoRI site of GST fusion vector pGEX-3×(Amersham-Pharmacia, Piscataway, N.J.). All of these CML66 fragments were fused in frame to the C-terminus of GST protein after cloning into the EcoRI site of the GST expression vector pGEX-3×and were further examined by DNA sequencing before transformation into the BL-21 strain of the E. coli. The GST and the full-length fusion protein GST-CML66 (25F1-25C, ORF1) were purified according to the manufacturer's protocols (Amersham-Pharmacia) or with B-per Bacterial Protein Extraction Reagent (Pierce, Rockford, Ill.).

Recombinant proteins expressed in transformed E. coli were subjected to 10-12% SDS-PAGE with Tris-Glycine buffer and transferred onto nitrocellulose filters in 20% methanol in Tris-Glycine buffer. Proteins on the blots were visualized as previously described (16).

### Example 11

### Quantitation of IgG Response to CML66 in Normal Donors and Patients with Cancer

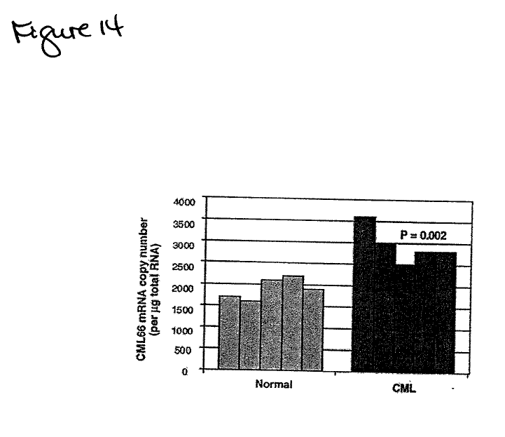

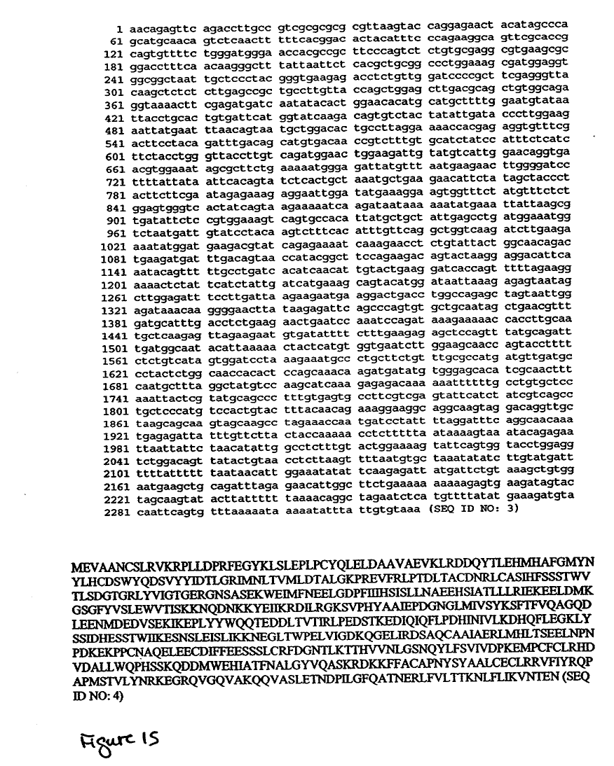

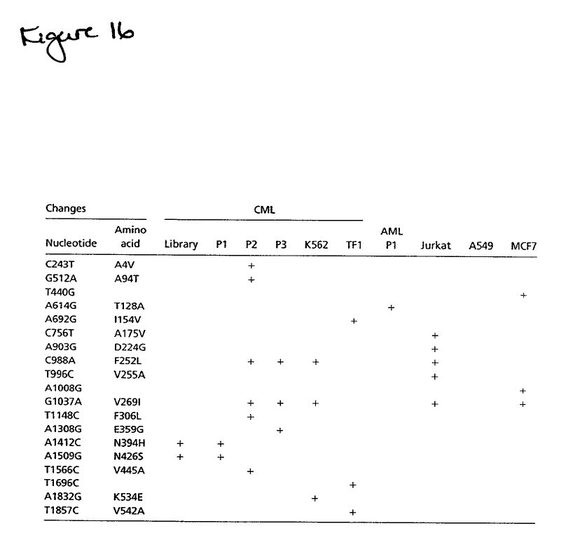

The CML66 ELISA was also used to detect and quantitate the levels of specific IgG antibody in sera obtained from normal donors and patients with different malignancies. In this assay, antibody reactivity with purified GST-CML66 was compared to reactivity with purified GST. As summarized in FIG. 12, reactivity was not detected in sera from normal donors (n=10) but specific CML66 reactivity was detected in patients with lung cancer (3 of 16 patients), melanoma (8 of 23 patients) and prostate cancer (20 of 39 patients). The highest level of reactivity was observed in the patient with CML known to have specific antibody by Western blot. In each instance where reactivity against GST-CML66 was greater than reactivity against GST, ELISA reactivity was blocked by prior incubation of sera with excess purified GST-CML66. These results confirm the specificity of the response to CML66 in these patients and indicate that CML66 is capable of eliciting a humoral immune response in patients with a variety of solid tumors.

ELISA plates (VWR Scientific, West Chester, Pa.) were coated with 50 μl of purified recombinant protein at 5 μg/ml in coating buffer (PBS+0.05% sodium azide) overnight at 4° C. (16). Plates were washed with PBS with 0.05% Triton X-100, and blocked overnight at 4° C. with 200 μl/well of 2% nonfat milk with 0.05% Triton X-100. 50 μl/well patient sera was added to a final dilution of 1:1000, and incubated at room temperature for 3 hours. The procedure for detection of specific IgG antibody has been described previously (16).

### REFERENCES

1. Clift, R. A., Buckner, C. D., Appelbaum, F. R., Bryant, E., Beannan, S. I., Peterson, F. B., Fisher, L. D., Anasetti, C., Beatty, P., Bensinger, W. I., Doney, K., Hill, R. S., McDonald, G. B., Martin, P., Meyers, J., Sanders, J., Singer, J., Stewart, P., Sullivan, K. M., Witherspoon, R., Storb, R., Hansen, J. A. & Thomas, E. D. (1991) Blood 77, 1660.

2. Clift, R. A., Buckner, C. D., Thomas, E. D., Bensinger, W. I., Bowden, R., Bryant, E., Deeg, H. J., Doney, K. C., Fisher, L. D., Hansen, J. A., Martin, P., McDonald, G. B., Sanders, J. E., Schoch, G., Singer, J., Storb, R., Sullivan, K. M., Witherspoon, R. P. & Appelbaum, F. R. (1994) Blood 84, 2036-2043.

3. Horowitz, M. M., Gale, R. P., Sondel, P. M., Goldman, J. M., Kersey, J., Kolb, H. J., Rimm, A. A., Ringden, O., Rozman, C., Speck, B. & et al., (1990) Blood 75, 555-62.

4. Sullivan, K. M., Weiden, P. L., Storb, R., Witherspoon, R. P., Fefer, A., Fisher, L., Buckner, C. D., Anasetti, C., Appelbaum, F. R., Badger, C., Beatty, P., Bensinger, W., Berenson, R., Bigelow, C., Cheever, M., Clift, R., Deeg, H., Doney, K., Greenberg, P., Hansen, J., Hill, R., Loughran, T., Martin, P., Neiman, P., Petersen, F., Sanders, J., Singer, J., Stewart, P. & Thomas, E. (1989) Blood 73, 1720-8.

5. Kolb, H. J., Schattenberg, A., Goldman, J. M., Hertenstein, B., Jacobsen, N., Arcese, W., Ljungman, P., Ferrant, A., Verdonck, L., Niederwieser, D. & et al., (1995) Blood 86, 2041-50.

6. Porter, D., Roth, M., McGarigle, C., Ferrara, J. & Antin, J. (1994) New Engl J Med 330, 100-106.

7. Sehn, L. H., Alyea, E. P., Weller, E., Canning, C., Lee, S., Ritz, J., Antin, J. H. & Soiffer, R. J. (1999) J Clin Oncol 17, 561-8.

8. Borrello, I., Sotomayor, E. M., Rattis, F. M., Cooke, S. K., Gu, L. & Levitsky, H. I. (2000) Blood 95, 3011-9.

9. Faber, L. M., van Luxemburg-Heijs, S. A., Willemze, R. & Falkenburg, J. H. (1992) J Exp Med 176, 1283-9.

10. Giralt, S., Estey, E., Albitar, M., van Besien, K., Rondon, G., Anderlini, P., O'Brien, S., Khouri, I., Gajewski, J., Mehra, R., Claxton, D., Andersson, B., Beran, M., Prezpiorka, D., Koller, C., Komblau, S., Korbling, M., Keating, M., Kantaijian, H. & Champlin, R. (1997) Blood 89, 4531-4536.

11. Slavin, S., Nagler, A., Naparstek, E., Kapelushnik, Y., Aker, M., Cividalli, G., Varadi, G., Kirschbaum, M., Ackerstein, A., Samuel, S., Amar, A., Brautbar, C., Ben-Tal, O., Eldor, A. & Or, R. (1998) Blood 91, 756-63.

12. Spitzer, T. R., McAfee, S., Sackstein, R., Colby, C., Toh, H. C., Multani, P., Saidman, S., Weyouth, D. W., Preffer, F., Poliquin, C., Foley, A., Cox, B., Andrews, D., Sachs, D. H. & Sykes, M. (2000) Biol Blood Marrow Transplant 6, 309-20.

13. Childs, R., Chemoff, A., Contentin, N., Bahceci, E., Schrump, D., Leitman, S., Read, E. J., Tisdale, J., Dunbar, C., Linehan, W. M., Young, N. S. & Barrett, A. J. (2000) N Engl J Med 343, 750-8.

14. Claret, E. J., Alyea, E. P., Orsini, E., Pickett, C. C., Collins, H., Wang, Y., Neuberg, D., Soiffer, R. J. & Ritz, J. (1997) J Clin Invest 100, 855-66.

15. Alyea, E. P., Soiffer, R. J., Canning, C., Neuberg, D., Schlossman, R., Pickett, C., Collins, H., Wang, Y., Anderson, K. C. & Ritz, J. (1998) Blood 91, 3671-80.

16. Wu, C. J., Yang, X. F., McLaughlin, S., Neuberg, D., Canning, C., Stein, B., Alyea, E. P., Soiffer, R. J., Dranoff, G. & Ritz, J. (2000) J Clin Invest 106, 705-14.

17. Sahin, U., Tureci, O., Schmnitt, H., Cochlovius, B., Johannes, T., Schmits, R., Stenner, F., Luo, G., Schobert, I. & Pfreundschuh, M. (1995) Proc Natl Acad Sci USA 92, 11810-3.

18. Old, L. J. & Chen, Y. T. (1998) J Exp Med 187, 1163-7.

19. Soiffer, R., Lynch, T., Mihm, M., Jung, K., Rhuda, C., Schmollinger, J. C., Hodi, F. S., Liebster, L., Lam, P., Mentzer, S., Singer, S., Tanabe, K. K., Cosimi, A. B., Duda, R., Sober, A., Bhan, A., Daley, J., Neuberg, D., Parry, G., Rokovich, J., Richards, L., Drayer, J., Berns, A., Clift, S., Dranoff, G. et al., (1998) Proc Natl Acad Sci USA 95,13141-6.

20. Yang, X. F., Weber, G. F. & Cantor, H. (1997) Immunity 7, 629-39.

21. Kozak, M. (1996) Mamm Genome 7, 563-74.

22. Lucas, S., De Smet, C., Arden, K. C., Viars, C. S., Lethe, B., Lurquin, C. & Boon, T. (1998) Cancer Res 58, 743-52.

23. Francke, U. (1994) Cytogenet Cell Genet 65, 206-18.

24. Konig, J., Teubel, W., Romijn, J., Schroder, F. & Hagemeijer, A. (1996) Hum Pathol 27, 720-727.

25. Chang, G., Tapsi, N., Steenbeek, M., Blok, L., van Weerden, W., van Al, D., Eussen, B., van Steenbrugge, G. & Brinkmann, A. (1999) Int J Cancer 83, 506-511.

26. Jager, E., Nagata, Y., Gnjatic, S., Wada, H., Stockert, E., Karbach, J., Dunbar, P. R., Lee, S. Y., Jungbluth, A., Jager, D., Arand, M., Ritter, G., Cerundolo, V., Dupont, B., Chen, Y. T., Old, L. J. & Knuth, A. (2000) Proc Natl Acad Sci USA 97, 4760-5.

27. Jager, E., Jager, D., Karbach, J., Chen, Y. T., Ritter, G., Nagata, Y., Gnjatic, S., Stockert, E., Arand, M., Old, L. J. & Knuth, A. (2000) J Exp Med 191, 625-30.

28. Zarour, H. M., Storkus, W. J., Brusic, V., Williams, E. & Kirkwood, J. M. (2000) Cancer Res 60, 4946-52.

29. Stockert, E., Jager, E., Chen, Y. T., Scanlan, M. J., Gout, I., Karbach, J., Arand, M., Knuth, A. & Old, L. J. (1998) J Exp Med 187, 1349-54.

30. Eibl, B., Schwaighofer, H., Nachbaur, D., Marth, C., Gachter, A., Knapp, R., Bock, G., Gassner, C., Schiller, L., Petersen, F. & Niederwieser, D. (1996) Blood 88, 1501-8.

31. Kolb et al., (1995) Blood :86:2041.

32. Porter et al., (1994) N Engl J Med. 330:100-6.

33. Alyea et al., (1998) Blood 91:3671-80.

34. Zhou and Deutscher (1997) J. Bacteriol. 179:4391-5.

35. Benard (1998) Mol. Cell Biol. 18:2688-96.

36. Jungbluth et al., (2000) Br J Cancer 83:493-7.

Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating or delaying the onset of an malignancy-associated disorder, said method comprising administering to a subject in need thereof an antibody to the polypeptide selected from the group consisting of SEQ ID NO: 2 and SEQ ID NO: 4 in an amount sufficient to treat or prevent said malignancy-associated disorder in said subject.

2. The method of claim 1 wherein the subject is a human.

3. The method of claim 1 wherein the malignancy-associated disorder is selected from the group consisting of leukemia and solid tumors.

4. The method of claim 1 wherein the malignancy-associated disorder comprises chronic myelocytic leukemia.

5. A method for determining the presence of or predisposition to a disease associated with altered levels of SEQ ID NO: 2 or SEQ ID NO: 4 in a first mammalian subject, said method comprising:
(a) providing a protein sample from said first mammalian subject;
(b) providing a control protein sample from a second mammalian subject known not to have or be predisposed to said disease;
(c) measuring the amount of SEQ ID NO: 2 or SEQ ID NO: 4 polypeptide in said subject sample; and
(d) comparing the amount of SEQ ID NO: 2 or SEQ ID NO: 4 polypeptide in said subject protein sample to the amount of SEQ ID NO: 2 or SEQ ID NO: 4 polypeptide in said control protein sample,
wherein an alteration in the expression level of the SEQ ID NO: 2 or SEQ ID NO: 4 polypeptide in the first subject sample as compared to the control sample indicates the presence or predisposition to said disease.

6. A method for determining the presence of or predisposition to a disease associated with altered levels of the nucleic acid of SEQ ID NO: 1 or SEQ ID NO: 3 in a first mammalian subject, said method comprising:
(a) providing a nucleic acid sample from said first mammalian subject;
(b) providing a control nucleic acid sample from a second mammalian subject known not to have or be predisposed to said disease;
(c) measuring the amount of SEQ ID NO: 1 or SEQ ID NO: 3 in said subject sample; and
(d) comparing the amount of SEQ ID NO: 1 or SEQ ID NO: 3 in said subject nucleic acid sample to the amount of SEQ ID NO: 1 or SEQ ID NO: 3 in said control nucleic acid sample,
wherein an alteration in the expression level of SEQ ID NO: 1 or SEQ ID NO: 3 in the first subject sample as compared to the control sample indicates the presence or predisposition to said disease.

7. A method of treating a pathological state in a mammal, the method comprising administering to the mammal a SEQ ID NO: 2 or SEQ ID NO: 4 polypeptide in an amount sufficient to alleviate the pathological state, wherein the polypeptide has an amino acid sequence at least 95% identical to the SEQ ID NO: 2 or SEQ ID NO: 4 polypeptide, or a biologically active fragment thereof

8. A method of treating a pathological state in a mammal, the method comprising administering to the mammal an antibody to a SEQ ID NO: 2 or SEQ ID NO: 4 polypeptide in an amount sufficient to alleviate the pathological state.

9. The method of claim 8 wherein the pathological state is selected from the group consisting of leukemia and a solid tumor.

10. The method of claim 8 wherein the pathological state comprises chronic myelocytic leukemia.

11. An isolated polypeptide comprising an amino acid sequence selected from the group consisting of:
(a) a mature form of an amino acid sequence selected from the group consisting of SEQ ID NOS: 2 and 4;
(b) a variant of a mature form of an amino acid sequence selected from the group consisting of SEQ ID NOS: 2 and 4, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS: 2 and 4; and
(d) a variant of an amino acid sequence selected from the group consisting of SEQ ID NOS: 2 and 4, wherein one or more amino acid residues in said variant differs from the amino acid sequence of said mature form, provided that said variant differs in no more than 15% of amino acid residues from said amino acid sequence.

12. The polypeptide of claim 11, wherein said polypeptide comprises the amino acid sequence of a naturally-occurring allelic variant of an amino acid sequence selected from the group consisting SEQ ID NOS: 2 and 4.

13. The polypeptide of claim 11, wherein said allelic variant comprises an amino acid sequence that is the translation of a nucleic acid sequence differing by a single nucleotide from a nucleic acid sequence selected from the group consisting of SEQ ID NOS: 1 and 3.

14. The polypeptide of claim 11, wherein the amino acid sequence of said variant comprises a conservative amino acid substitution.

15. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2 and 4;
(b) a variant of a mature form of an amino acid sequence selected from the group consisting of SEQ ID NOS: 2 and 4, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2 and 4;
(d) a variant of an amino acid sequence selected from the group consisting SEQ ID NOS: 2 and 4, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2 and 4, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

16. The nucleic acid molecule of claim 15, wherein the nucleic acid molecule comprises the nucleotide sequence of a naturally-occurring allelic nucleic acid variant.

17. The nucleic acid molecule of claim 15, wherein the nucleic acid molecule encodes a polypeptide comprising the amino acid sequence of a naturally-occurring polypeptide variant.

18. The nucleic acid molecule of claim 15, wherein the nucleic acid molecule differs by a single nucleotide from a nucleic acid sequence selected from the group consisting of SEQ ID NOS: 1 and 3.

19. The nucleic acid molecule of claim 15, wherein said nucleic acid molecule comprises a nucleotide sequence selected from the group consisting of:
(a) a nucleotide sequence selected from the group consisting of SEQ ID NOS: 1 and 3;
(b) a nucleotide sequence differing by one or more nucleotides from a nucleotide sequence selected from the group consisting of SEQ ID NOS: 1 and 3, provided that no more than 20% of the nucleotides differ from said nucleotide sequence;
(c) a nucleic acid fragment of (a); and
(d) a nucleic acid fragment of (b).

20. The nucleic acid molecule of claim 15, wherein said nucleic acid molecule hybridizes under stringent conditions to a nucleotide sequence chosen from the group consisting SEQ ID NOS: 1 and 3, or a complement of said nucleotide sequence.

21. The nucleic acid molecule of claim 15, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and p1 (c) a nucleic acid fragment of (a) or (b).

22. A vector comprising the nucleic acid molecule of claim 15.

23. The vector of claim 22, further comprising a promoter operably-linked to said nucleic acid molecule.

24. A cell comprising the vector of claim 23.

25. An antibody that binds immunospecifically to the polypeptide of claim 11.

26. The antibody of claim 25, wherein said antibody is a monoclonal antibody.

27. The antibody of claim 25, wherein the antibody is a humanized antibody.

28. A method for determining the presence or amount of the polypeptide of claim 11 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

29. A method for determining the presence or amount of the nucleic acid molecule of claim 15 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

30. The method of claim 29 wherein presence or amount of the nucleic acid molecule is used as a marker for cell or tissue type.

31. The method of claim 30 wherein the cell or tissue type is cancerous.

32. The method of claim 31 wherein the cancer is selected from leukemia and a solid tumor.

33. The method of claim 32 wherein the leukemia is chronic myelocytic leukemia.

34. A method of identifying an agent that binds to a polypeptide of claim 11, the method comprising:
(a) contacting said polypeptide with said agent; and
(b) determining whether said agent binds to said polypeptide.

35. The method of claim 34 wherein the agent is a cellular receptor or a downstream effector.

36. A method for identifying an agent that modulates the expression or activity of the polypeptide of claim 11, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

37. A method for modulating the activity of the polypeptide of claim 11, the method comprising contacting a cell sample expressing the polypeptide of said claim with a compound that binds to said polypeptide in an amount sufficient to modulate the activity of the polypeptide.

38. A pharmaceutical composition comprising the polypeptide of claim 11 and a pharmaceutically-acceptable carrier.

39. A pharmaceutical composition comprising the nucleic acid molecule of claim 15 and a pharmaceutically-acceptable carrier.

40. A pharmaceutical composition comprising the antibody of claim 25 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.
